SEQUENCE LISTING (22 sequences)

SEQ ID NO: 1 - DNA, 132 bp, Meloidogyne chitwoodi
atgtttactaattcaattaaaaatttaattatttatttaatgcctttaatggttactttaatgcttttgtctgtctcatttgtggatgcaggcaaaaagcctagtgggccaaatcctggaggaaataattga

SEQ ID NO: 2 - DNA, 153 bp, Meloidogyne chitwoodi
atgtcccaatcaattaaaaatttaataatatttttaatttattttattattaatttaattattttatctgttacttttgtggattcagcaaaaggaaagaaagaaagcagtggaccatcactaggtggaaatgataataatgatggtcgctaa

SEQ ID NO: 3 - protein, 43 aa, Meloidogyne chitwoodi
MFTNSIKNLIIYLMPLMVTLMLLSVSFVDAGKKPSGPNPGGNN

SEQ ID NO: 4 - protein, 50 aa, Meloidogyne chitwoodi
MSQSIKNLIIFLIYFIINLIILSVTFVDSAKGKKESSGPSLGGNDNNDGR

SEQ ID NO: 5 - RNA, 152 nt, Meloidogyne chitwoodi
augucccaaucaauuaaaaauuuaauaauauuuuuaauuuauuuuauuauuaauuuaauuauuuuaucuguuacuuuuguggauucagcaaaaggaagaaagaaagcaguggaccaucacuagguggaaaugauaauaaugauggucgcuaa

SEQ ID NO: 6 - RNA, 152 nt, Meloidogyne chitwoodi
uuagcgaccaucauuauuaucauuuccaccuagugaugguccacugcuuucuuucuuccuuuugcugaauccacaaaaguaacagauaaaauaauuaaauuaauaauaaaauaaauuaaaaauauuauuaaauuuuuaauugauugggacau

SEQ ID NO: 7 - DNA, 152 bp, Meloidogyne chitwoodi
ttagcgaccatcattattatcatttccacctagtgatggtccactgctttctttcttccttttgctgaatccacaaaagtaacagataaaataattaaattaataataaaataaattaaaaatattattaaatttttaattgattgggacat

SEQ ID NO: 8 - DNA, 28 bp, Artificial Sequence
  Synthetic oligonucleotide primer
gtttactaattcaattaaaaatttaatt

SEQ ID NO: 9 - DNA, 26 bp, Artificial Sequence
  Synthetic oligonucleotide primer
caattatttcctccaggatttggccc

SEQ ID NO: 10 - DNA, 18 bp, Artificial Sequence
  Synthetic oligonucleotide primer
gcgcaaggatgacacgca

SEQ ID NO: 11 - DNA, 20 bp, Artificial Sequence
  Synthetic oligonucleotide primer
ggctgagttatttttttctg

SEQ ID NO: 12 - DNA, 26 bp, Artificial Sequence
  Synthetic oligonucleotide primer
ggggtcaaaccctttggcacgtctgg

SEQ ID NO: 13 - DNA, 25 bp, Artificial Sequence
  Synthetic oligonucleotide primer
gcgggtgatctcgactgagttcagg

SEQ ID NO: 14 - DNA, 30 bp, Artificial Sequence
  Synthetic oligonucleotide primer
gatatttaaattaaattatattcttctaaa

SEQ ID NO: 15 - DNA, 27 bp, Artificial Sequence
  Synthetic oligonucleotide primer
gctttattcaatttatttttatttatt

SEQ ID NO: 16 - DNA, 31 bp, Artificial Sequence
  Synthetic oligonucleotide primer
ttattttatctgttacttttgtggattcagc

SEQ ID NO: 17 - DNA, 27 bp, Artificial Sequence
  Synthetic oligonucleotide primer
gcgaccatcattattatcatttccacc

SEQ ID NO: 18 - DNA, 20 bp, Artificial Sequence
  Synthetic oligonucleotide primer
ttcgcaagacccttcctcta

SEQ ID NO: 19 - protein, 26 aa, Artificial Sequence
  Synthetic oligonucleotide primer
CTTCTTCGTCTTACACATCACTTGTC

SEQ ID NO: 20 - protein, 14 aa, Artificial Sequence
  Synthetic peptide motif
KRXVPXGPNPLHNR

SEQ ID NO: 21 - DNA, 21 bp, Artificial Sequence
  Synthetic oligonucleotide primer
atcgggagcggcgataccgta

SEQ ID NO: 22 - DNA, 21 bp, Artificial Sequence
  Synthetic oligonucleotide primer
gacgctattcggctatgactg

(19) United States
(12) Patent Application Publication
(10) Pub. No.: US 2015/0259700 A1
(43) Pub. Date: Sep. 17, 2015

(54) Transgenic Plants With RNA Interference-Mediated Resistance Against Root-Knot Nematodes

(71) Applicant: WASHINGTON STATE UNIVERSITY, PULLMAN, WA (US)

(72) Inventors: Axel A. Elling, Pullman, WA (US); Charles R. Brown, Pullman, WA (US)

(21) Appl. No.: 14/626,070

(22) Filed: Feb. 19, 2015

Related U.S. Application Data
(60) Provisional application No. 61/948,761, filed on Mar. 6, 2014.

(51) Int. Cl. C12N 15/82; C12N 15/113

(52) U.S. Cl. CPC C12N 15/8285; C12N 15/113; C12N 2310/141; C12N 2310/531

(57) ABSTRACT

Transgenic plants that are stably resistant to the nematode Meloidogyne chitwoodi are provided, as are methods of making such transgenic plants. The transgenic plants (such as potatoes) are genetically engineered to express interfering RNA that targets the Meloidogyn effector protein Mc16D10L.

### CROSS-REFERENCE TO RELATED APPLICATIONS

This application claims benefit of U.S. provisional patent application 61/948,761, filed Mar. 6, 2014, the complete contents of which is hereby incorporated by reference.

### STATEMENT OF FEDERALLY SPONSORED RESEARCH AND DEVELOPMENT

This invention was made with government support under grant number 58-5354-1-467 awarded by United States Department of Agriculture/Agricultural Research Service. The government has certain rights in the invention.

### SEQUENCE LISTING

This application includes as the Sequence Listing the complete contents of the accompanying text file “Sequence.txt”, created Feb. 16, 2015, containing 6.14 kilobytes, hereby incorporated by reference.

### DESCRIPTION

### BACKGROUND OF THE INVENTION

1. Field of the Invention

The invention generally relates to stable transgenic plants that are resistant to the nematode Meloidogyne chitwoodi. In particular, the invention provides stable transgenic plants (such as potatoes) in which the Mc16D10L gene, which encodes a putative M. chitwoodi effector protein, is targeted using RNA interference.

2. BACKGROUND OF THE INVENTION

The potato (Solanum tuberosum L.) is the most important non-cereal food crop worldwide and makes up the staple diet of over 1 billion people. Global potato production per year has been estimated at over 374 million metric tons in 2012, but varies widely on a country-by-country basis and depends on local growing conditions and agricultural practices. The highest yields are reached in Western Europe and the USA at about 50 t/ha, whereas average yields in Africa and most of Asia are no higher than 13 t/ha. Potato production has increased dramatically over the past two decades and developing countries now account for most of the market volume, which underscores the importance of this crop for global food security.

Potatoes are attacked by a wide variety of diseases and pests that can reduce tuber yield and quality significantly. Plant-parasitic nematodes are a major threat to potato production worldwide, with root-knot (Meloidogyne spp.) and cyst nematodes (Globodera spp.) being the most widespread and causing most of the damage. In the Pacific Northwest of the USA, which is the leading potato growing area and accounts for more than half of the country's total production, the Columbia root-knot nematode (Meloidogyne chitwoodi Golden et al.) is the most common and significant nematode threat to sustainable potato cultivation. M. chitwoodi is not only a problem in the USA but has also been found in potato growing regions in Europe, Mexico, Argentina and South Africa, making it a threat in some of the world's most important potato production areas. M. chitwoodi causes tuber quality defects and can render entire shipments unmarketable. It has been estimated that as little as one juvenile per 250 g soil at the beginning of the growing season can lead to a total loss of marketability at harvest. To limit spread, M. chitwoodi has been designated as a quarantine pest, which has a significant impact on the international trade of potato tubers.

Meloidogyne spp. are obligate parasites that depend on their host plant for survival. The pathogenic part of the life cycle of root-knot nematodes begins with the second-stage juveniles (J2), which are the infective life stage and invade plant roots, rhizomes and tubers. The J2 migrate intercellularly through host tissue until they become sedentary and induce the formation of giant cells, which constitute the nematodes' feeding site and sole source of nutrition. The exact mechanisms that lead to the formation and maintenance of giant cells are unknown, but generally it is thought that secretory proteins from the nematode esophageal gland cells, i.e., effectors, play a key role in the underlying processes. After a feeding site has been established, the J2 increase in size and undergo subsequent molts into third- and fourth-stage juveniles (J3, J4). After a final molt they develop into adult females or males. The females remain sedentary, but the males regain their motility, exit the plant tissue and fertilize the females. Many Meloidogyne spp. are parthenogenic and not all species form males. M. chitwoodi reproduces by facultative meiotic parthenogenesis. Adult females deposit eggs into egg masses, a gelatinous matrix that protects eggs from desiccation. Inside the eggs, first-stage juveniles (J1) develop and molt into J2, which hatch under favorable conditions that are mostly dictated by soil moisture and temperature.

Nematode control in most potato growing areas is based on routine applications of synthetic nematicides. This practice not only is costly but also potentially harmful to the environment because some products have been linked to negative effects on the atmosphere's ozone layer. Host resistance against root-knot nematodes would be an ideal control strategy, but is difficult to achieve. In spite of the extremely rich genetic resources found in Solanum sect. Petota, which consists of wild and cultivated potatoes and includes up to 232 tuber-bearing and non-tuber-bearing species, exploiting this genetic diversity and introducing M. chitwoodi resistance into cultivated potato (S. tuberosum ssp. tuberosum) has proven to be challenging. In spite of over two decades of breeding efforts no commercial potato cultivar with resistance to M. chitwoodi is available to date. The R_{Mc1} gene from S. bulbocastanurn has been used to develop advanced S. tuberosum breeding clones, but M. chitwoodi populations are very variable and some isolates are able to overcome the R_{Mc1} gene, presenting a significant problem for traditional resistance breeding. Furthermore, hairy nightshade (S. sarrachoides Sendtn.), a common weed in potato producing areas in the western USA and a host for M, chitwoodi, was found to undermine resistance under greenhouse and field conditions when it co-occurs with potato breeding lines carrying the R_{Mc1} resistance gene.

There is a need in the art to develop new methods for preventing damage by M. chitwoodi in crops susceptible to infection by this nematode.

### SUMMARY OF THE INVENTION

Data presented herein establishes for the first time that the protein encoded by the M. chitwoodi gene Mc16D10L is an effector protein of M. chitwoodi, and that resistance to M. chitwoodi is increased in plants that are genetically engineered to stably express interfering RNA that prevents or at least decreases expression of the Mc16D10L effector protein in the transgenic plants. Nematodes that infect such transgenic plants produce as many as 70% fewer eggs and egg masses than nematodes that infect comparable control plants. Significantly, nematodes that infect the transgenic plants described herein also transmit these characteristics to their offspring so that the RNAi effect is maintained over several M. chitwoodi generations. Thus, plants other than those which are genetically engineered as described herein also benefit from the technology.

Other features and advantages of the present invention will be set forth in the description of invention that follows, and in part will be apparent from the description or may be learned by practice of the invention. The invention will be realized and attained by the compositions and methods particularly pointed out in the written description and claims hereof.

Provided herein are stable transgenic plants that are resistant to Meloidogyne chitwoodi. The stable transgenic plants are genetically engineered to express dsRNA complementary to a Meloidogyn effector gene, e.g. the Meloidogyn effector gene Mc16D10L. The dsRNA may be shRNA. The stable transgenic plants may be, for example, potatoes, carrots, tomatoes, alfalfa or black salsify. In some aspects, the stable transgenic plant is a potato plant.

Also provided are transgenic plant cells or transgenic plant parts comprising dsRNA. One strand of the dsRNA is complementary to mRNA encoding a Meloidogyn effector protein. In some aspects, the Meloidogyn effector protein is Mc16D10L. In some aspects, the dsRNA is shRNA and the one strand is a guide strand. The stable transgenic plants may be, for example, potatoes, carrots, tomatoes, alfalfa or black salsify. In some aspects, the stable transgenic plant is a potato plant.

Also provided are methods of producing a plant that is stably resistant to Meloidogyne chitwoodi. The methods comprise a step of genetically engineering the plant to contain and express dsRNA which comprises one strand that is complementary to mRNA encoding a Meloidogyn effector protein. In some aspects, the Meloidogyn effector protein is Mc16D10L. In some aspects, the dsRNA is shRNA and the one strand is a guide strand. The stable transgenic plants may be, for example, potatoes, carrots, tomatoes, alfalfa or black salsify. In some aspects, the stable transgenic plants are potato plants.

### BRIEF DESCRIPTION OF THE DRAWINGS

FIG. 1A and B. Sequence alignment of 16D10 orthologs from M. incognita (Mi16D10) and M, chitwoodi (Mc16D10L) at the (A) nucleotide and (B) amino acid level. SEQ ID NO: 1=Mi16D10 nucleotide sequence; SEQ ID NO: 2=Mi16D10 protein sequence; SEQ ID NO: 3=Mc16D10L nucleotide sequence; SEQ ID NO: 4=Mc16D10L protein sequence.

FIG. 2 A-D. In situ hybridization of Mc16D10L in different M. chitwoodi life stages. Spatial expression pattern of Mc16D10L in M. chitwoodi eggs (A), infective J2 (B) and parasitic J2 (C). In infective and parasitic J2, Mc16D10L was expressed specifically in the subventral esophageal glands. (D) Negative control using a sense probe. Scale bar equals 20 μm.

FIG. 3 Relative transcript abundance of Mc16D10L in different M. chitwoodi life stages. Using the transcript level of Mc16D10L in eggs as a reference, Mc16D10L was upregulated significantly (1.87-fold on a log₁₀ scale) in infective J2 (J2). In parasitic J2 (pJ2), mixed J3/J4 parasitic juveniles and adult females (F), Mc16D10L was downregulated significantly (−0.72, −1 and −1.6-fold on a log₁₀ scale, respectively). Each bar represents the log₁₀ transformed mean of qRT-PCR reactions run in triplicates with standard errors. Letters indicate statistically significant differences using a Student's t-test (P<0.05).

FIGS. 4A and B. Reproductive success of M. chitwoodi on transgenic A. thaliana expressing pART27(16D10i-2). (A) Number of egg masses per plant at 35 DAI, and (B) number of eggs per plant at 55 DAI in Columbia-0 wild type (COL), transgenic empty pART27 vector control (E2), and transgenic pART27(16D10i-2) (D1, D2, D4) plants. Each bar represents the mean of nine plants per independent line and timepoint with standard errors. Letters indicate statistically significant differences using a Student's t-test (P<0.05).

FIG. 5A-D. Reproductive success of M. chitwoodi on transgenic potato expressing pART27(16D10i-2). (A) Number of egg masses per plant at 35 DAI, (B) number of eggs per plant at 55 DAI, (C) number of egg masses per gram root fresh weight at 35 DAI and (D) number of eggs per gram root fresh weight at 55 DAI in cv. Désirée wild type (DES), transgenic empty pART27 vector control (E29), and transgenic pART27(16D10i-2) (D54, D56, D57) plants. Each bar represents the mean of ten plants per independent line and timepoint with standard errors. Letters indicate statistically significant differences using a Student's t-test (P<0.05).

FIG. 6A-C. Production of small RNAs in transgenic Arabidopsis and potato plants. (A) U6 small nuclear RNA (snRNA) loading control, (B) small RNA and (C) total RNA in Arabidopsis Columbia-0 wild type (COL), transgenic empty pART27 vector control (E2), and transgenic pART27(16D10i-2) (D1, D2, D4), and in potato cv. Désirée wild type (DES) and transgenic pART27(16D10i-2) (D54, D56, D57) plants.

FIGS. 7A and B. Relative fold change of Mc16D10L transcript level in second-generation M. chitwoodi from transgenic potato lines. Relative transcript abundance of Mc16D10L in M. chitwoodi eggs (A) and infective J2 (B). Eggs were harvested from transgenic potato lines expressing the silencing construct pART27(16D10i-2) and used either directly for qRT-PCR or allowed to hatch infective J2 in modified Baermann pans. Potato lines included cv. Désirée wild type (DES), transgenic empty pART27 vector control (E29) and transgenic pART27(16D10i-2) (D54, D56, D57). Each bar represents the mean of qRT-PCR reactions run in triplicates with standard errors. Letters indicate statistically significant differences using a Student's t-test (P<0.05).

FIG. 8. Southern blots showing copy numbers of 16D10i-2 in stable transgenic potato lines. Lines based on cv. Désirée include DES, wild type; E29, empty vector pART27; D56, D57, D12, D42, transformed with pART27(16D10i-2). Lines based on cv. Russet Burbank include RB, wild type; E34, empty vector pART27; D5, D25, D20, D16, transformed with pART27(16D10i-2). Lines based on advanced breeding line PA99N82-4 include 82-4, wild type; E12, empty vector pART27; D17, D53, D55, D2, transformed with pART27(16D10i-2).

FIG. 9A-C. Northern blots for stable transgenic potato lines. A: 16D10i-2-specific small RNAs hybridizing with probe 16D10; B: U6 small nuclear RNA (snRNA) loading control; C: Total RNA loading control. DES, wild type; E29, empty vector pART27; D56, D57, D12, D42, transformed with pART27(16D10i-2), all with cv. Désirée as genetic background. RB, wild type; E34, empty vector pART27; D5, D25, D20, D16, transformed with pART27(16D10i-2), all with cv. Russet Burbank as genetic background. 82-4, wild type; E12, empty vector pART27; D17, D53, D55, D2, transformed with pART27(16D10i-2), all with advanced breeding line PA99N82-4 as genetic background.

FIG. 10A-D. Reproductive success of M. chitwoodi WAMC1 on transgenic potato lines expressing 16D10i-2. A: Number of egg masses per plant at 35 days after inoculation (DAI); B: Number of egg masses per gram root fresh weight at 35 DAI; C: Number of eggs per plant at 55 DAI; D: Number of eggs per gram root fresh weight at 55 DAI. DES, wild type; E29, empty vector pART27; D56, D57, D12, D42, transformed with pART27(16D10i-2), all with cv. Désirée as genetic background. RB, wild type; E34, empty vector pART27; D5, D25, D20, D16, transformed with pART27(16D10i-2), all with cv. Russet Burbank as genetic background. 82-4, wild type; E12, empty vector pART27; D17, D53, D55, D2, transformed with pART27(16D10i-2), all with advanced breeding line PA99N82-4 as genetic background. Each bar represents the mean of 10 plants per independent line and timepoint with standard errors. Letters indicate statistically significant differences using a Student's t-test (P<0.05).

FIG. 11A-D. Reproductive success of M. chitwoodi Roza on transgenic potato lines expressing 16D10i-2 in a PA99N82-4 genetic background. A: Number of egg masses per plant at 35 days after inoculation (DAI); B: Number of egg masses per gram root fresh weight at 35 DAI; C: Number of eggs per plant at 55 DAI; D: Number of eggs per gram root fresh weight at 55 DAI. 82-4, wild type; E12, empty vector pART27; D17, D53, D55, D2, transformed with pART27(16D10i-2), all with advanced breeding line PA99N82-4 as genetic background. Each bar represents the mean of 10 plants per independent line and timepoint with standard errors. Letters indicate statistically significant differences using a Student's t-test (P<0.05).

FIG. 12A-D. Pathogenicity and reproductive success of M. chitwoodi offspring from potato lines with and without the 16D10i-2 RNAi transgene. A: Number of egg masses per plant at 35 days after inoculation (DAT). (B) Number of egg masses per gram root fresh weight at 35 DAI; C: Number of eggs per plant at 55 DAI; D: Number of eggs per gram root fresh weight at 55 DAI. M. chitwoodi WAMC1 inoculum was harvested from transgenic potato cv. Désirée lines E29 (empty vector control) and D56 (carrying pART27(16D10i-2)), resulting in four different treatments: empty vector potato line E29 inoculated with M. chitwoodi eggs collected from line E29 (pE29-eE29); empty vector line E29 inoculated with M. chitwoodi eggs collected from 16D10i-2 line D56 (pE29-eD56), 16D10i-2 line D56 inoculated with M. chitwoodi eggs collected from line empty vector line E29 (pD56-eE29) and 16D10i-2 line D56 inoculated with M. chitwoodi eggs collected from line D56 (pD56-eD56), where ‘p’ stands for plant and ‘e’ stands for eggs. M. chitwoodi isolate used was WAMC1. Each bar represents the mean of 10 plants per independent line and timepoint with standard errors. Letters indicate statistically significant differences using a Student's t-test (P<0.05).

FIG. 13A-D. Relative fold change of Mc16D10L transcript level in M. chitwoodi J2 offspring from different potato-nematode combinations. Eggs were collected from infection assays pE29, pD56, pE29-eE29, pE29-eD56, pD56-eE29 and pD56-eD56 and used to hatch J2. A: qRT-PCR showing relative fold changes of Mc16D10L transcript level in J2. Each bar represents the mean of qRT-PCR reactions run in triplicates with standard errors. Letters indicate statistically significant differences using a Student's t-test (P<0.05); B: Northern blot showing Mc16D10L transcript level in J2. (C) Northern blot for ITS2 expression as control; D: Agarose gel showing total RNA loading control. Genetic background of all potato lines used was cv. Désirée and M. chitwoodi isolate was WAMC1. pE29, empty vector line E29 inoculated with M. chitwoodi collected from wild type tomatoes cv. Rutgers; pD56, 16D10i-2 line D56 inoculated with M. chitwoodi collected from wild type tomatoes cv. Rutgers; pE29-eE29, empty vector line E29 inoculated with M. chitwoodi eggs collected from line E29; pE29-eD56, empty vector line E29 inoculated with M. chitwoodi eggs collected from 16D10i-2 line D56; pD56-eE29, 16D10i-2 line D56 inoculated with M. chitwoodi eggs collected from line empty vector line E29; pD56-eD56, 16D10i-2 line D56 inoculated with M. chitwoodi eggs collected from line D56, where ‘p’ stands for plant and ‘e’ stands for eggs. Eggs harvested to hatch J2 for qRT-PCR and northern blots are from the same plants analyzed for pathogenicity of M. chitwoodi offspring as shown in FIG. 5.

FIG. 14. Schematic illustration of shRNA production and folding.

### DETAILED DESCRIPTION

Data presented in the present disclosure demonstrates that the M. chitwoodi gene Mc16D10L is an effector protein of M. chitwoodi. Based on this discovery, interfering RNA (iRNA) technology has been used to create genetically engineered plants which display stable enhanced resistance to M. chitwoodi. The transgenic plants stably express iRNA that targets Mc16D10L mRNA. M. chitwoodi nematodes that infect such transgenic plants produce as many as 70% fewer eggs and egg masses than nematodes which infect comparable control plants. Without being bound by theory, it is believed that the infecting nematodes ingest the iRNA and that the ingested iRNA acts within the nematode to inhibit translation of Mc16D10L mRNA, thereby preventing expression (and thus activity) of the Mc16D10L effector protein. Interestingly, nematodes which infect the transgenic plants described herein transmit these characteristics (inhibition of expression and activity of the Mc16D10L effector protein) to their offspring so that, even if the offspring have never infected a transgenic plant that is stably resistant to M. chitwoodi, they still produce reduced levels of eggs and egg masses. In other words, the offspring nematodes are also indirectly “stably genetically engineered”. This trait permits plants other than those which are genetically engineered as described herein to also benefit from the technology.

The following definitions are used throughout.

“RNA interference” (RNAi) refers to a biological process in which RNA molecules inhibit gene expression, typically by causing the destruction of specific (targeted) mRNA molecules.

“Interfering RNA” (iRNA) is a class of double-stranded RNA molecules, e.g. about 15-30 base pairs in length (e.g. about 15, 16, 17, 18, 29, 20, 21, 22, 23, 24, 25, 26, 27, 28, 29 or 30 base pairs), and typically about 20-25 bps in length. In the RNA interference (RNAi) pathway, iRNA interferes with the expression of specific genes with complementary nucleotide sequences. iRNA functions by binding to transcribed mRNA that encodes a protein of interest (a targeted protein), thereby causing the mRNA to be broken down. As a result, the mRNA is not translated and the encoded protein is not produced or expressed. Herein, a “targeted” gene or protein is a gene or protein whose expression (and hence activity) is prevented in this manner. iRNA (e.g. as a dsRNA construct) may be introduced directly into a cell, or may be generated within the cell by processing of larger dsRNA molecules such as shRNA.

A small hairpin RNA or short hairpin RNA (shRNA) is a sequence of RNA that folds into a dsRNA structure that includes a base-paired stem and a tight hairpin turn at one end of the molecule. shRNA can be used to silence target gene expression via RNA interference (RNAi). Expression of shRNA in cells is typically accomplished by delivery of plasmids or through viral or bacterial vectors which encode the shRNA, which is cleaved in the cell to form iRNA.

By “resistant” we mean that a plant displays a decrease in symptoms of infection associated with or characteristic of nematode infection, compared to a comparable control plant, e.g. a wild type plant, or a plant that has not been genetically engineered to contain and express iRNA that targets Mc16D10L gene expression (but that may or may not have been otherwise selected or genetically engineered to have other desirable traits).

A “decrease in symptoms” refers to a level of symptoms that is at least about 30% (or more) less than (below) the level of comparable control plants, e.g. about 30, 40, 45, 50, 55, 60, 65, 70, 75, 80, 85, 90, 95 or 100% lower. Generally, the level of symptoms in a resistant plant is at least about 50-80% lower, and may be e.g. at least about 60-70% lower, than those displayed by comparable control plants.

The “symptoms of infection” that are used as indicators of the level of infection include but are not limited to: number of eggs or egg masses produced in or on an infected plant; egg masses per gram root fresh weight; number of eggs per gram root fresh weight; number of nematodes (of any stage, e.g. juveniles, adults, etc.) associated with (e.g. on or in) a plant; amount of damage to a plant (e.g. decrease in growth rate, increase in number or extent of lesions or damaged portions of a plant, decrease in weight e.g. root and/or foliage weight; decrease in foliage growth, growth rate and/or increase in non-healthy appearing leaves, changes of Mc16D10L expression (e.g. mRNA production), etc.), compared to a statistically significant number of comparable plants that are not infected with M. chitwoodi.

“M. chitwoodi” refers to both race 1 and race 2 of the nematode.

A genetically engineered (or “transformed”) plant or plant cell (or other organism, e.g. a nematode or nematode cell) that is “stably transgenic” is one which contains and expresses at least one heterologous nucleic acid sequence, and which transmits the at least one heterologous nucleic acid sequence to offspring, i.e. the offspring inherits the at least one heterologous nucleic acid sequence. Generally, phenotypic traits associated with expression of the at least one heterologous nucleic acid are expressed in both the transgenic parent plant and in the offspring over at least several generations. The heterologous nucleic acid may be maintained outside the chromosome of the transformed cell or organism (epigentically) or by incorporation into a chromosome of the transformed cell or organism. Stable integration (insertion) into the chromosome may be preferable, and a heterologous nucleic acid sequence (e.g. DNA) may be inserted at a single chromosomal location or a multiple (one or more) sites.

A “heterologous” or “exogenous” nucleic acid or protein sequence is one that is present in a genetically engineered plant or other genetically engineered organism but that is not present in wild type counterparts of the genetically engineered plant/organism in nature. Genetically engineered plants/organisms harboring heterologous nucleic acids or proteins may be referred to as “transgenic”, “genetically engineered”, etc. In the present disclosure, a DNA sequence encoding iRNA that targets Mc16D10L mRNA is considered to be a “heterologous” nucleic acid sequence.

The present disclosure describes transgenic plants that are genetically engineered to contain and express DNA sequences encoding iRNA that is complementary to mRNA encoding the effector protein Mc16D10L. Methods of making such transgenic plants are also provided. The general procedure for making transgenic plants that harbor such sequences is known in the art and involves identifying a protein, the expression of which is unwanted or undesirable, and identifying the gene that encodes the protein. Protein expression is well-known to occur via transcription of a gene into mRNA, and iRNA technology targets the transcribed mRNA to prevent its translation into the unwanted protein.

The RNAi pathway is initiated by the enzyme Dicer, which cleaves long exogenous double-stranded RNA (dsRNA) molecules (e.g. dsRNA produced due to genetic engineering of the organism) into short double stranded fragments of ˜20 nucleotide iRNAs. Each iRNA is unwound into two single-stranded RNAs (ssRNAs), a passenger strand and a guide strand. The sense (passenger) strand is degraded and the antisense (guide) strand is incorporated into an RNA-induced silencing complex (RISC). While all details of the RNAi mechanism have not been elucidated, one well-studied outcome is post-transcriptional gene silencing, which occurs when the guide strand in the RISC pairs with a complementary sequence in an mRNA molecule that is encountered in the cell. The precise mechanism is not fully understood, but it is believed that the antisense (guide) strand “directs” RISC to mRNA that has such a complementary sequence. In the case of perfect complementarity between the guide strand and the mRNA, catalytic enzyme components of the RISC complex, argonaute proteins, cleave the mRNA strand, preventing its translation. In the case of imperfect complementarity (e.g. imperfect base pairing or a complementary sequence that is shorter than the full length mRNA), RISC represses translation of the mRNA, possibly by steric hindrance of the translation machinery of the cell. Either way, the outcome is a drastic decrease in (silencing of) the expression of a targeted gene. In some instances, RNAi may not totally abolish expression of a targeted gene, and is referred to as a “knockdown” procedure (to distinguish it from “knockout” procedures in which expression of a gene is entirely eliminated).

In the present invention, plants are genetically engineered to contain and express DNA that encodes dsRNA comprising a guide strand that is complementary to mRNA encoding Mc16D10L, and a passenger strand that is complementary to the guide strand. Generally, the dsRNA is provided as shRNA which is cleaved via the dicer pathway as described above. Binding of the guide strand to RISC results in capture of Me16D10L-encoding mRNA and its translation is prevented. Reduced expression of the Mc16D10L effector protein attenuates that ability of M. chitwoodi nematodes to infect and reproduce in the transgenic plants. As a result, the number of eggs and egg masses is decreased, and damage to the transgenic plants is decreased and/or fewer plants are damaged.

The disclosure provides vectors for use in producing the transgenic plants described herein. The vectors generally comprise a DNA sequence encoding an RNA molecule comprising i) an RNA sequence encoding the Mc16D10L protein (a sense sequence) and ii) an antisense sequence that is complementary to the sense sequence, the two sequences being located in the same molecule and separated by a short sequence that does not base pair with the sense and antisense sequences, but which, when the RNA molecule folds via base pairing between the sense and antisense sequences, forms a loop at one end of the folded molecule.

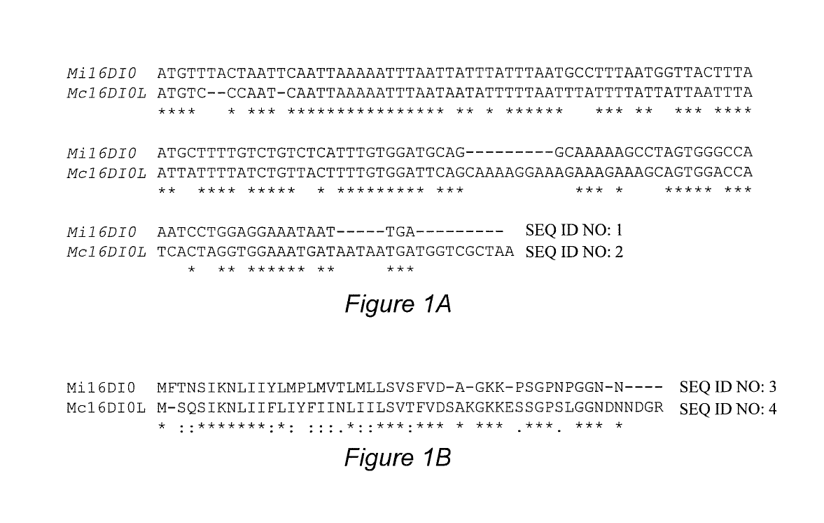

The DNA sequence of the gene encoding Mc16D10L is presented herein as SEQ ID NO: 3 (see FIG. 1 and below):

(SEQ ID NO: 2)
5′-atgtcccaatcaattaaaaatttaataatatttttaatttattttat
tattaatttaattattttatctgttacttttgtggattcagcaaaaggaa
gaaagaaagcagtggaccatcactaggtggaaatgataataatgatggtc
gctaa-3′.

The corresponding sense strand of mRNA is presented below as SEQ ID NO: 5:

(SEQ ID NO: 5)
5′-AUGUCCCAAUCAAUUAAAAAUUUAAUAAUAUUUUUAAUUUAUUUUAU
UAUUAAUUUAAUUAUUUUAUCUGUUACUUUUGUGGALTUCAGCAAAAGGA
AGAAAGAAAGCAGUGGACCAUCACUAGGUGGAAAUGAUAAUAAUGAUGGU
CGCUAA-3′.

The corresponding antisense strand for SEQ ID NO: 5 is SEQ ID NO: 6:

(SEQ ID NO: 6)
5′-UUAGCGACCAUCAUUAUUAUCAUUUCCACCUAGUGAUGGUCCACUGC
UUUCUUUCUUCCUUUUGCUGAAUCCACAAAAGUAACAGAUAAAAUAAUUA
AAUUAAUAAUAAAAUAAAUUAAAAAUAUUAUUAAAUUUUUAAUUGAUUGG
GACAU-3′.

A suitable DNA sequence to encode SEQ ID NO: 6 is SEQ ID NO: 7:

(SEQ ID NO: 7)
5′-TTAGCGACCATCATTATTATCATTTCCACCTAGTGATGGTCCACTGC
TTTCTTTCTTCCTTTTGCTGAATCCACAAAAGTAACAGATAAAATAATTA
AATTAATAATAAAATAAATTAAAAATATTATTAAATTTTTAATTGATTGG
GACAT-3′.

In some aspects, a vector that is used to transgenically modify a plant comprises SEQ ID NO: 2 and SEQ ID NO: 7, or portions thereof that are complementary to each other, and the RNA sequence that is encoded by the vector comprises SEQ ID NO: 5 and SEQ ID NO: 6, or portions (segments) thereof that are complementary to each other. If the encoded RNA sequence is an shRNA, the DNA also encodes an intervening loop-forming sequence that is not complementary to either of SEQ ID NO: 5 and SEQ ID NO: 6, or to at least one of SEQ ID NO: 5 and SEQ ID NO: 6. Generally, if complementary segments of SEQ ID NOS: 5 and 6 are encoded, the segments (which will e.g. form a stem sequence in shRNA) will be from about 15-150 base pairs in length (e.g. about 15, 20, 25, 30, 35, 40, 45, 50, 55, 60, 65, 70, 75, 80, 85, 90, 95, 100, 105, 110, 120, 125, 130, 135, 140, 145 or 150 base pairs. Typically the segment is on the order of about 15-35 base pairs in length e.g. about 15, 16, 17, 18, 19, 20, 21, 22, 23, 24, 25, 26, 27, 28, 29, 30, 31, 32, 33, 34 or 35 contiguous base pairs, and generally will be about 19-29 base pairs in length e.g. 19, 20, 21, 22, 23, 24, 25, 26, 27, 28 or 29 base pairs. In some aspects, the number of base pairs in the “stem” portion of the shRNA is about 22-23 complementary base pairs.

In other aspects, the sequence that is encoded by the vector is an shRNA comprising sequences that have at least about 50, 55, 60, 65, 70, 75, 80, 85, 90, 95 or more (e.g. 96, 97, 98 or 99) % homology to SEQ ID NO: 5 and SEQ ID NO: 6. Generally, such modified sequences are 100% homologous to each other so that proper base pairing is preserved in the dsRNA construct (e.g. shRNA). In yet other aspects, the two complementary sequences in the shRNA are from about 90-100% homologous to portions of SEQ ID NO: 5 and SEQ ID NO: 6, e.g. to contiguous, complementary portions of SEQ ID NO: 5 and SEQ ID NO: 6, and comprise e.g. at least about 20, 30, 40, 50, 60, 70, 80, 90, 100, 110, 120, 130, 140 or 150 base pairs.

Those of skill in the art will recognize that the precise structure of an encoded shRNA can vary. For example, the loop may be formed from sequences that are not present in either the sense or antisense strand and the sense and antisense strands may base pair precisely at the 5′ and 3′ ends. Alternatively, the loop may be formed from a sequence that is present in either the sense or the antisense strand, but for which the complementary sequence is not present in the opposite strand. This short segment thus cannot base pair (although the rest of the molecule can) and forms a loop. For example, in an exemplary embodiment, an shRNA comprises a first sequence of 22 or 23 nucleotides fully complementary to a sequence in the coding region of a target gene, and a second sequence directly following the first sequence, wherein the second sequence is fully complementary to the sequence of the first 17 or 18 nucleotides counted from 5′ end of the first sequence. In addition, a dinucleotide overhang may be present at one end of the stem, e.g. typically the 3′ end of the stem. In some aspects, the antisense strand is positioned 5′ to the loop; in other aspects, the antisense strand is positioned 3′ to the loop. Loop sizes may vary being e.g. as small as 1 or 4 nt, or larger, e.g. from about 3-10 bases, and are generally from about 4 to about 8 bases in length, e.g. the loop size may vary and may be about 1, 2, 3, 4, 5, 6, 7, 8, 9, or 10 bases.

In some aspects, what is provided herein is transgenic plants or plant cells comprising chromosomal DNA comprising an exogenous (non-native) DNA sequence inserted at one or multiple locations of the chromosomal DNA, the exogenous DNA sequence encoding the complementary RNA sequences as set forth in SEQ ID NOS: 5 and 6, or a segment of SEQ ID NO: 5 and a segment of SEQ ID NO: 6, the segments being complementary to each other, together with an intervening loop-encoding sequence between the complementary sequences, and optionally, a 5′ overhang is also encoded.

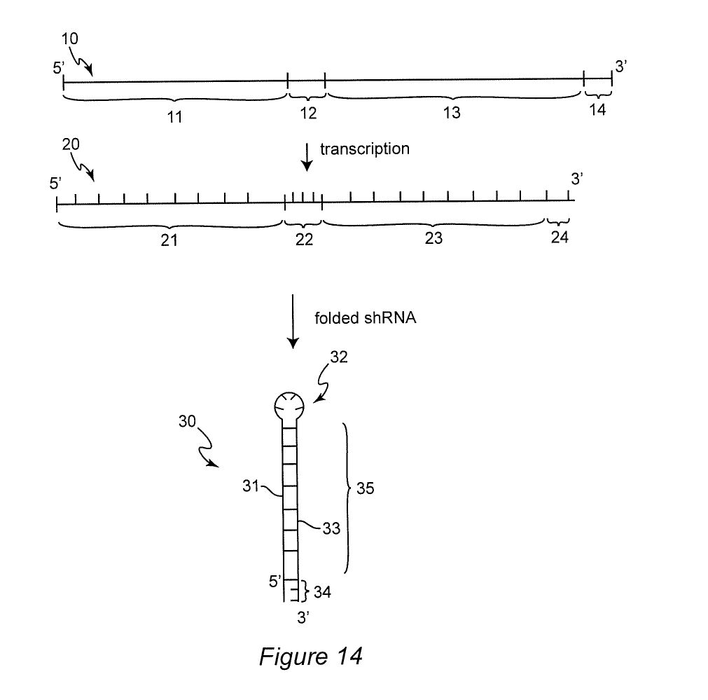

A schematic depiction of an exemplary DNA insertion is provided in FIG. 14. Depicted are: DNA sequence 10 comprising DNA sequence encoding SEQ ID NO: 5 (or encoding a portion thereof) 11; loop-encoding DNA sequence 12; DNA sequence encoding SEQ ID NO: 6 (or encoding a portion thereof) 13; and optional DNA sequence encoding a 3′ dinucleotide overhang 14. Also depicted is transcribed unfolded RNA molecule 20, which comprises single strand RNA segment 21 (which is SEQ ID NO: 5 or a portion thereof); ssRNA loop segment 22; ssRNA segment 23 (which is SEQ ID NO: 6 or a portion thereof complementary to the portion of SEQ ID NO: 5); and optional 3′ overhang 24 (e.g. about one, two or three nucleotides). Also depicted is folded RNA molecule 30. As can be seen the molecule contains loop 32 and double strand stem 35, with stem 35 comprising base-paired sequences 31 and 33 (SEQ ID NO: 5 or a portion thereof and SEQ ID NO: 6 or a portion thereof, respectively); and 3′ overhang 34. Cleavage of shRNA molecule 30 results in separation of the stem 35 sequences, and antisense guide sequence SEQ ID NO: 6 (or a portion thereof) 33 associates with an RNA-induced silencing complex (RISC) in which it serves to bind (capture) Mc16D10L mRNA and cause its destruction, or inhibition of its translation.

DNA sequences encoding such shRNA molecules, whether or not they are present in a vector, are also encompassed by the invention. In addition, vectors which comprise the DNA sequences are encompassed. The vectors which encode the dsRNAs typically include a promoter or promoter region that is able to achieve robust RNA expression. Exemplary promoters for expression of a transgene include plant promoters such as CaMV 35S, CaMV 19S, nos, Adh, sucrose synthase, a-tubulin, actin, cab, PEPCase, and those associated with the R gene complex. Tissue specific promoters such as root cell promoters and tissue specific enhancers, terminators, etc. are also contemplated to be particularly useful, as are inducible promoters such as ABA- and turgor-inducible promoters. In certain aspects, a promoter for use according to the invention is a ePCISV, TubA, eFMV, FMV, e35S, 35S or Ract1 promoter.

Suitable vectors for use in generating transgenic plants as described herein include but are not limited to: plasmids and various plant specific vectors such as Agrobacterium tumefaciens. Vectors can be delivered by any suitable means, e.g. using gene guns, electroporaton, or microinjection.

In some aspects, the vector is the pART27 binary plasmid plant expression vector originally described by Gleave (Plant Molecular Biology 20:1203-1207, 1992). The expression cartridge of the primary cloning vector, pART7, comprises of cauliflower mosaic virus Cabb B-JI isolate 35S promoter, a multiple cloning site and the transcriptional termination region of the octopine synthase gene. The entire cartridge can be removed from pART7 as a Not I fragment and introduced directly into the binary vector, pART27, recombinants being selected by blue/white screening for β-galactosidase. pART27 carries the RK2 minimal replicon for maintenance in Agrobacterium, the ColE1 origin of replication for high-copy maintenance in Escherichia coli and the Tn7 spectinomycin/streptomycin resistance gene as a bacterial selectable marker. The organizational structure of the T-DNA of pART27 was constructed taking into account the right to left border, 5′ to 3′ model of T-DNA transfer. The T-DNA carries the chimeric kanamycin resistance gene (nopaline synthase promoter-neomycin phosphotransferase-nopaline synthase terminator) distal to the right border relative to the lacZ′ region. The constructs of sense DNAs as described herein may be made, for example, by cloning a cDNA insert into a pART7 plasmid, which is then cut by NotI enzyme and the 35S-Insert-OCS 3′ UTR is put into a pART27 binary plasmid plant expression vector. The presence and integrity of the transgenic constructs may be verified by restriction digestion and DNA sequencing. Other modified versions of pART27 may also be employed, e.g. the binary vector pART29, a modified pART27 vector that contains the Arabidopsis thaliana ubiquitin 3 (UBQ3) promoter instead of the nos5′ promoter and no lacZ sequences (see also U.S. Pat. No. 8,809,629 to Forster, et al. and U.S. Pat. No. 8,481,813 to Eady, et al., the entire contents of both of which are herein incorporated by reference.

Alternative methods of creating the transgenic plants include utilizing expression cassettes for shRNA interference that are designed such that the sense sequence and antisense sequence do not correspond to a nucleic acid sequence encoding the polypeptide of interest. Instead, the sense and antisense sequences flank a loop sequence that comprises a nucleotide sequence corresponding to all or part of the nucleic acid sequence encoding the polypeptide of interest. Thus, the loop region determines the specificity of the RNA interference. Alternatively, transcriptional gene silencing (TGS) can be accomplished through use of shRNA molecules in which an inverted repeat of the hairpin shares sequence identity with the promoter region of a nucleic acid encoding the polypeptide of interest to be silenced. Processing of the shRNA produces short RNAs that can interact with a homologous promoter region (e.g. the promoter region for the gene encoding the Mc16D10L protein) and may trigger degradation or methylation to result in silencing of the gene.

In yet further alternatives, dsRNA can be supplied without genetic engineering. One approach is to add dsRNAs to irrigation water. The molecules are then absorbed into the plants' vascular system and nematodes which infect the plants ingest the dsRNA and are inhibited. Another approach involves spraying dsRNA like a conventional pesticide. This approach allows faster adaptation to resistance, but requires low cost sources of RNAs.

Alternatively, techniques such as TALENs (Transcription activator-like effector nucleases) and CRISPRs (clustered regularly interspaced short palindromic repeats) may be utilized to inactivate the gene encoding Mc16D10L in a plant of interest Inhibition of the expression and/or activity of the Mc16D10L protein may be carried out by any suitable means known in the art, and all plants in which the expression and/or activity of Mc16D10L protein is inhibited (either prevented entirely, or decreased) are encompassed by the invention.

M. chitwoodi has a wide host range among several plant families, including crop plants and common weed species. The transgenic plants described herein may be of any type that is susceptible to infection by M. chitwoodi, including plants that are good or very good hosts and consequently are damaged by the nematode, and those which are moderate or poor hosts but can harbor and maintain the nematode. Such plants include but are not limited to: potatoes (Solanum tuberosum), tomatoes (Lycopersicon esculentum), peas (Pisum sativum), Phaseolus vulgaris (the common bean, string bean, field bean, flageolet bean, French bean, garden bean, haricot bean, pop bean, or snap bean), Scorzonera hispanica (black salsify or Spanish salsify, also known as black oyster plant, serpent root, viper's herb, viper's grass or simply scorzonera), lucerne, carrots and sugarbeet (Beta vulgaris var. saccharifera) are good hosts. Various cereals such as barley (Hordeum vulgare), maize (Zea mays), oats (Avena sativa), wheat (Triticum aestivum), Eragrostis curvula, Arachis hypogaea and various Poaceae (grasses and weeds) are relatively poor hosts but will maintain the nematode, with rye and summer wheat supporting relatively high populations of the nematode. Lucerne (Medicago sativa) is a good host for race 2 but not for race 1, whereas carrots (Daucus carota) are a non-host for race 2 but a good host for race 1.

Moderate to poor hosts occur in the Brassicaceae, Cucurbitaceae, Fabaceae, Lamiaceae, Liliaceae, Urnbelliferae and Vitaceae families. Brassica rapes, Eragrostis tef and Lolium multiflorum support high populations.

Plants that are genetically engineered as described herein include any that are susceptible to infection by M. chitwoodi, whether or not the infection leads to damage to the plant and/or whether or not the damage has commercial consequences. The plants may be susceptible to infection by natural routes of transmission (e.g. through contaminated soil or contact with infected plants), or may be susceptible to infection only with human intervention, e.g. under laboratory conditions.

In some aspects, the plants (e.g. crops) that are genetically engineered are not good hosts for the nematode, but can maintain the nematode so that genetically engineering resistance as described herein reduces or prevents entirely their ability to maintain M. chitwoodi. Such moderate to poor hosts, when genetically engineered, can be valuable when used in crop rotation schemes since they will not maintain high levels of the nematode, and may decrease or eliminate the M. chitwoodi burden in soil in which they are planted so that subsequently planted crops (e.g. potatoes) will suffer less damage, whether or not they are also resistant. Further, nematodes that infect poor or moderate hosts still transfer the properties of less infectivity to nematode offspring, further reducing the ill effects on subsequent crops. This disclosure thus also provides methods of crop rotation that involve alternating the planting of a first crop that is a moderate or poor host for M. chitwoodi but which is genetically engineered to be resistant to the nematode, with a second crop of interest that is a good host in order to lessen damage to the second crop of interest, whether or not the latter is also resistant to the nematode.

In one aspect of the invention, the plant that is genetically engineered as described herein is a potato plant. “Potatoes” include starchy, tuberous crops from the perennial nightshade Solanum tuberosum, of which there are close to 4000 different varieties, any of which may be genetically engineered as described herein. Exemplary categories include but are not limited to: russets, reds, whites, yellows (also called Yukons) and purples. Exemplary varieties (cultivars) of these which may be genetically engineered as described herein include but are not limited to: Adirondack Blue, Adirondack Red, Agata, Almond, Alpine Russet, Alturas, Amandine, Annabelle, Anya, Arran Victory, Atlantic, Austrian Crescent, Avalanche, Bamberg, Bannock Russet, Belle de Fontenay, BF-15, Bildtstar, Bintje, Blazer Russet, Blue Congo, Bonnotte, British Queens, Cabritas, Camota, Canela Russet, Cara, Carola, Chelina, Chiloé, Cielo, Clavela Blanca, Désirée, Estima, Fianna, Fingerling, Flava, French Fingerling, German Butterball, Golden Wonder, Goldrush, Home Guard, Innovator, Irish Cobbler, Irish Lumper, Jersey Royal, Kennebec, Ken's Pink, Kestrel, Keuka Gold, King Edward, Kipfler, Lady Balfour, Langlade, Linda potato, Marcy, Marfona, Mans Piper, Marquis, Megachip, Monalisa, Nicola, Norgold Russet, Pachacoña, Pike, Pink Eye, Pink Fir Apple, Primura,

Ranger Russet, Ratte, Record, Red La Soda, Red Norland, Red Pontiac, Rooster, Russet Burbank, Russet Norkotah, Selma, Shepody, Sieglinde, Silverton Russet, Sirco, Snowden, Spunta, Up to date, Stobrawa, Superior, Villetta Rose, Vivaldi, Vitelotte, Yellow Finn and Yukon Gold.

The plant that is genetically engineered as described herein may be a plant that is not a potato plant, but generally will be a plant that is a host for M. chitwoodi. In some aspects, the plant is a host that supports growth and or numbers of the nematode sufficient to cause commercially relevant damage to the plant, e.g. tomatoes, peas, beans, black salsify, carrots and Lucerne, etc. In other aspects, the plant supports growth and or numbers of the nematode that are not sufficient to cause commercially relevant damage to the plant, but the plant would, in the absence of being genetically engineered, maintain a reservoir of the nematode that can e.g. then infect plants located in proximity to the reservoir, or plants which are later planted in the same or adjacent soil, or plants which come into contact with reservoir plants, e.g. during harvest, shipping, storage, etc.

Encompassed herein are whole plants and portions of plants, as well as all life stage forms of plants, including, for example, bulbs, tubers, corms, rhizomes, flowers, inflorescences, cones, calyx, fruits (inc. pods), leaves, stems (above ground), stalks, shoots, roots, true seeds (e.g. grains), embryos, etc., as well as individual cells that are genetically modified as described herein, or which are obtained from a plant that has been genetically modified as described herein. Plant tissue of any type is also encompassed.

Before exemplary embodiments of the present invention are described in greater detail, it is to be understood that this invention is not limited to particular embodiments described, as such may, of course, vary. It is also to be understood that the terminology used herein is for the purpose of describing particular embodiments only, and is not intended to be limiting.

Where a range of values is provided, it is understood that each intervening value between the upper and lower limit of that range (to a tenth of the unit of the lower limit) is included in the range and encompassed within the invention, unless the context or description clearly dictates otherwise. In addition, smaller ranges between any two values in the range are encompassed, unless the context or description clearly indictates otherwise.

Unless defined otherwise, all technical and scientific terms used herein have the same meaning as commonly understood by one of ordinary skill in the art to which this invention belongs. Representative illustrative methods and materials are herein described; methods and materials similar or equivalent to those described herein can also be used in the practice or testing of the present invention.

All publications and patents cited in this specification are herein incorporated by reference as if each individual publication or patent were specifically and individually indicated to be incorporated by reference, and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual dates of public availability and may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as support for the recitation in the claims of such exclusive terminology as “solely,” “only” and the like in connection with the recitation of claim elements, or use of a “negative” limitations, such as “wherein [a particular feature or element] is absent”, or “except for [a particular feature or element]”, or “wherein [a particular feature or element] is not present (included, etc.) . . . ”.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### EXAMPLES

### Example 1

RNA interference-mediated down regulation of effector gene Mc16D10L confers resistance against Columbia root-knot nematode in Arabidopsis thaliana and potato

Transgenic approaches, and in particular the use of RNA interference (RNAi), could be an attractive alternative for nematode control. RNAi was first described in the model nematode Caenorhabditis elegans and it has since been found that it is involved in a wide variety of biological processes in plants and animals. The phenomenon is based on double-stranded RNA (dsRNA), which is processed by Dicer, a ribosome III-like enzyme, into shorter small interfering RNA (siRNA) fragments of about 21 nt. The siRNAs are loaded into a multisubunit complex named RISC (RNA induced silencing complex), which catalyzes the degradation of complementary mRNA. Plant-parasitic nematodes are known to take up host cytoplasm during the infection process, making them vulnerable to host-derived compounds, including dsRNA and siRNA. This dependency has been exploited recently by engineering transgenic plant tissue that overexpresses dsRNA that is complementary to nematode genes. However, plant-mediated RNAi technology to create nematode resistance has been restricted largely to the model plant Arabidopsis thaliana and Agrobacterium rhizogenes-induced transgenic hairy roots. Stable transformation of agriculturally important crops to implement host-mediated RNAi against nematodes has been achieved only in tobacco (Nicotiana tabacum L.) and soybean (Glycine max (L.) Merr.) to date, and with greatly varying levels of resistance. In some of these cases, nematode genes were targeted that are conserved in other animals, and could prove problematic when implemented in the field. Targeting genes that are specific to plant-parasitic nematodes to eliminate off-target effects might provide a better option if this technology is to be employed in crops. The effector gene 16D10 has shown great promise as an RNAi target in other Meloidogyne spp., but it is unknown whether this gene exists in M. chitwoodi and whether it can be used to create stable resistance against this nematode, especially in agronomically important crops. Therefore, the objective of this study was to test whether creating stable transgenic lines of A. thaliana and potato that overexpress dsRNA complementary to the Meloidogyne-specific effector gene 16D10 leads to resistance against M. chitwoodi.

### Experimental Procedures

### Nematode Maintenance and Extraction

M. chitwoodi isolate WAMC1 was maintained on tomato (S. lycopersicum L.) cv. Rutgers under greenhouse conditions (Humphreys-Pereira and Elling, 2013). Tomato plants were grown in autoclaved sand. Three months after inoculation, M. chitwoodi eggs and parasitic life stages were harvested from tomato roots. To extract eggs, roots were cut into small pieces and agitated for 3 min in a 0.5% sodium hypochlorite solution (Hussey and Barker, 1973). The root suspension was poured over a set of nested test sieves (#20, #200 and #500 from top to bottom) and eggs were collected from the #500 sieve. To purify samples, the M. chitwoodi egg suspension was divided into 50 mL tubes (20 mL per tube) and mixed with 20 mL 70% sucrose. Ten mL water were carefully placed on top of the suspension and the tubes were immediately centrifuged for 3 min at 375×g in a clinical centrifuge. After centrifuging, eggs were collected from the interface and rinsed on a #500 test sieve. To hatch infective J2, purified eggs were incubated in a modified Baermann pan with sterile water at room temperature for four days; hatched infective J2 were collected by centrifugation (Dinh et al., 2014). To extract parasitic nematode life stages, root pieces from which eggs were extracted were blended in tap water and decanted over a set of four nested test sieves (#60, #120, #350 and #500 from top to bottom). Each sieve was rinsed for 2 min with tap water. M. chitwoodi females were collected from the #120 sieve, parasitic J3 and J4 from the #350 sieve and parasitic J2 from the #500 sieve, respectively. To purify parasitic nematode stages, samples were transferred to 50 mL tubes and 10 mL of 25% MgSO₄.7H₂O were added to the bottom of each tube using a transfer pipet. Tubes were centrifuged as above and parasitic life stages were collected from the interface and rinsed on a #500 sieve. To ensure purity of parasitic life stages and to remove remaining plant debris, samples were purified further by manually removing contaminants under a Stemi 2000C stereomicroscope (Zeiss, Jena, Germany). All developmental stages were rinsed either in DEPC-treated water before being stored at −80° C. for use in qRT-PCR or resuspended in 4% formaldehyde in phosphate buffered saline for in situ hybridizations (see below).

Nematode RNA Purification and Cloning of Mc16D10L Full-Length cDNA

Total RNA was extracted from frozen tissue of all M. chitwoodi life stages using the PerfectPure RNA Fibrous Tissue kit (5Prime, Gaithersburg, Md., USA) following the manufacturer's instructions. Approximately 500 ng purified RNA of each life stage was used as template to synthesize cDNA using the ADVANTAGE® RT-for-PCR kit (Clontech, Mountain View, Calif., USA) according to the manufacturer's protocol. Eighty μL DEPC-treated water were added to each 20 μL reverse transcription product and samples were stored at −80° C. To clone full-length cDNA of Mc16D10L, primers Mc16D10-F and Mc16D10-R were designed based on previously published M. chitwoodi EST sequence CD418743 (Roze et al., 2008). Using primers Mc16D10-F and Mc 16D10-R and PHUSION® polymerase (New England Biolabs, Ipswich, Ma., USA), Mc16D10L was amplified from 5 μL cDNA of infective J2 in a MASTERCYCLER® pro thermal cycler (Eppendorf, Hamburg, Germany) with the following PCR program: 94° C. for 5 min; 40 cycles of 94° C. for 30 s, 52° C. for 45 s and 72° C. for 30 s, followed by 72° C. for 5 min. The PCR product was cloned into PGEM®-TEasy (Promega, Madison, Wis., USA), and transformed into E. coli DH5-alpha (New England Biolabs). The plasmid was recovered using the GENEJET™ Plasmid Miniprep kit (Thermo Scientific, Rochester, N.Y., USA), and sequenced (Elim Biopharm, Hayward, Calif., USA) to confirm the identity of the insert. Sequences were aligned using ClustalW and signal peptides were analyzed with SignalP 3.0 software.

### In Situ Hybridization

To determine the spatial and temporal expression patterns of Mc16D10L in M. chitwoodi, in situ hybridizations were conducted for all life stages. After amplifying the Mc16D10L template with primers Mc16D10-F and Mc16D10-R using PHUSION® polymerase (New England Biolabs), the sense and antisense probes were synthesized with the PCR DIG Probe Synthesis kit (Roche, Indianapolis, Ind., USA) following the manufacturer's instructions. Eggs, infective J2, J3, J4 and adult females that were fixed previously in 4% formaldehyde in phosphate buffered saline for 2 days at room temperature were cut at random with a razor blade, dehydrated and hybridized in 1.5 mL microcentrifuge tubes with sense (for negative control) and antisense probes at 37° C. for 16 h (Hussey et al., 2011). After incubation, residues were removed and probes detected following Hussey et al. (2011). Hybridization patterns were viewed and documented with an AxioObserver A1 inverted microscope equipped with differential interference contrast, AxioCam ICc1 digital camera, and ZEN imaging software (Zeiss). All experiments were replicated at least twice and showed similar results.

### Generation of Transgenic Arabidopsis and Potato

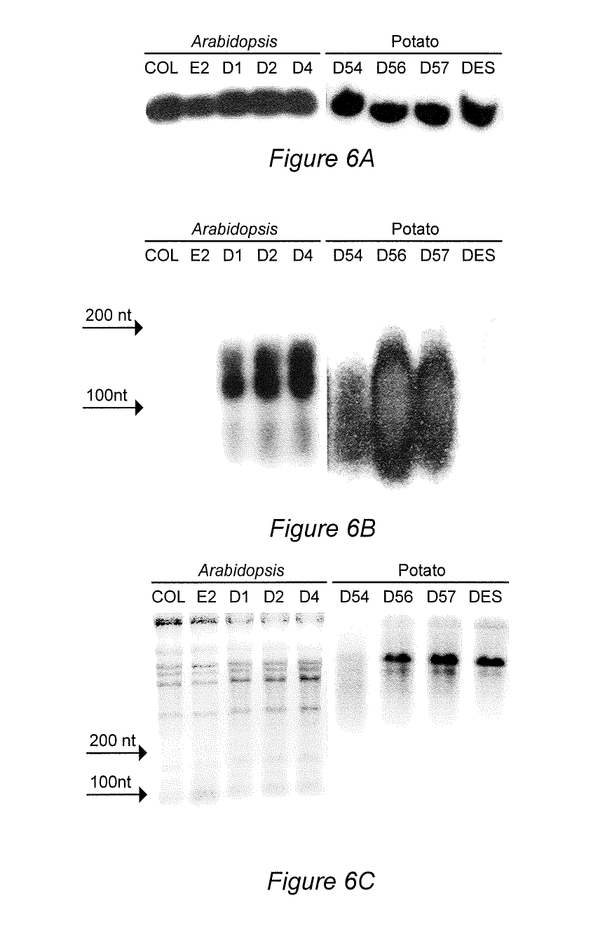

A. thaliana ecotype Columbia (Col-0) was used as wild type in infection assays described below and as genetic background for generating transgenic RNAi lines following the floral dip method (Clough and Bent, 1998). Using Agrobacterium tumefaciens strain GV3101, the empty binary vector pART27 and the silencing construct pART27(16D10i-2), which contained a pHANNIBAL RNAi cassette based on Mi16D10 (Huang et al., 2006a), were transformed into A. thaliana. For each construct, approximately 500 seeds from 24 individual plants were germinated on ½ strength Murashige and Skoog (MS) basal salts media (Caisson, North Logan, Utah, USA), supplemented with 3% D-sucrose, 50 μg/mL kanamycin sulfate and 0.5 g/L timentin, solidified with 0.6% Daishin agar at pH 5.7. Seedlings were maintained at 22° C. in a growth chamber with a 12 h photoperiod. Selection-resistant seedlings were confirmed by PCR for presence of the transgene, and for each construct, six seedlings were carefully transferred to individual pots filled with potting mix, and cultivated in the greenhouse (16 h daylight at 22° C. and 8 h nighttime at 20° C.). T₁ seeds were collected from five individual lines and about 50 T₁ seeds for each line were plated on selection media to ensure the presence of the transgene. One plant per line was grown in the greenhouse until maturity to collect T2 seeds, later plated to confirm transgenic phenotypes by kanamycin resistance and PCR. For all experiments described here, T₃ lines were used; these were confirmed by PCR, northern (FIG. 6) and Southern blots (not shown). Three transgenic RNAi lines (D1, D2, D4), one empty vector line (E2) and a wild type control line (Col-0) were used for infection assays.

The same constructs also were introduced into potato cv. Désirée. Internodal stem pieces were co-incubated with A. tumefaciens strain GV3101, carrying the empty pART27 or pART27(16D10i-2) binary vector on sterile CIM media (MS basal salts, 0.25 ppm folic acid, 0.05 ppm D-biotin, 2 ppm glycine, 0.5 ppm nicotinic acid, 0.5 ppm pyridoxine HCl, 0.4 ppm thiamine HCl, 0.01% myo-inositol, 3% D-sucrose, 1 ppm 6-benzylaminopurine, 2 ppm 1-naphthaleneacetic acid, 0.6% Daishin agar at pH 5.6) for three days in the dark at 19° C. (Brown et al., 1991b). For the empty pART27 lines, 200 internodal stem pieces were used for transformation, and 250 were used for pART27(16D10i-2), respectively. Stem segments were transferred every two weeks to sterile 3C5ZR media (MS basal salts, 0.5 ppm nicotinic acid, 0.5 ppm pyridoxine HCl, 1 ppm thiamine HCl, 0.01% myo-inositol, 3% D-sucrose, 0.5 ppm indole-3-acetic acid, 3 ppm zeatin ribose, 0.5 g/L timentin, 70 μg/mL kanamycin sulfate, 0.6% Daishin agar at pH 5.9) and maintained in a growth chamber 22° C. with a 12 h photoperiod (Sheerman and Bevan, 1988). Newly formed plantlets that regenerated from callus tissue on 3C5ZR plates were transferred to propagation media (MS basal salts, 3%

D-sucrose, 50 μg/mL kanamycin sulfate, 0.6% Daishin agar at pH 5.7) and maintained under the same growth conditions (55 plantlets for pART27 and 64 plantlets for pART27(16D10i-2)).

Regenerated plantlets were analyzed by PCR, northern (FIG. 6) and Southern blots to ensure presence of the transgene. For Southern blots, lines used include Arabidopsis Columbia-0 wild type (COL), transgenic empty pART27 vector control (E2) and transgenic pART27(16D10i-2) (D1, D2, D4), as well as potato cv. Désirée wild type (DES), transgenic empty pART27 vector control (E29) and pART27(16D10i-2) (D54, D56, D57). Fifteen μg genomic DNA of each Arabidopsis and potato line was digested with XbaI (New England Biolabs), loaded on a 0.8% agarose gel, transferred to a membrane and hybridized with a [α-³²P] dATP (MP Biochemicals) probe that was synthesized with primers 35S-F and OCS1 (Table 1). None of the wild type or empty vector control lines showed any signal. Lines D1 and D2 had multiple insertions of the transgene, whereas lines D4, D54, D56 and D57 had single insertions (not shown).

Three single-insertion pART27(16D10i-2) lines (D54, D56, D57), one empty vector line (E29) and a wild type control line (DES) were chosen for infection assays in the greenhouse. BLAST searches between the gene-specific region of pART27(16D10i-2) and Arabidopsis and potato ESTs and genome sequences did not yield any significant hits, suggesting that no in planta off-target silencing effects were to be expected.

### Infection Assays

For Arabidopsis, Col-0 wild type and transgenic (E2, D1, D2, D4) seeds were surface-sterilized and germinated in a growth chamber (22° C. with a 12 h photoperiod) on ½ strength MS media supplemented with 3% D-sucrose and 50 μg/mL kanamycin sulfate (for transgenic lines) and solidified with 0.6% Daishin agar at pH 5.7. After nine days, seedlings were transferred to individual 500 mL pots filled with SunShine Mix #1 potting mix (Sun Gro Horticulture, Agawam, Mass., USA), and grown for ten days under greenhouse conditions before being inoculated with 5,000 M. chitwoodi eggs per pot. For potato, single nodes of wild type cv. Désirée and transgenic (E29, D54, D56, D57) lines were cut from plants maintained in tissue culture and propagated in a growth chamber (22° C. with a 12 h photoperiod) on MS media supplemented with 3% D-sucrose and 50 μg/mL kanamycin sulfate (for transgenic lines) and solidified with 0.6% Daishin agar at pH 5.7 for one month. Following, plants were transferred to individual 500 mL pots filled with autoclaved sand, and cultivated under greenhouse conditions for ten days before being inoculated with 5,000 M. chitwoodi eggs per pot. Sand was used to facilitate cleaning of potato roots at the conclusion of the infection assay. Arabidopsis did not grow well in pure sand, hence the use of potting mix. Both Arabidopsis and potato plants were maintained in the greenhouse for the duration of the experiment, and watered twice per day with 20-10-20 NPK liquid fertilizer. Infection assays were designed as randomized complete block designs with nine replications for each Arabidopsis line and timepoint (35 and 55 DAI, respectively) and ten replications for each potato line and timepoint. At 35 DAI the roots were washed free of growth substrate and for potato, the root fresh weight was measured. Potting mix adhering to roots prevented reliable fresh root weight measurements in Arabidopsis and the weights had to be omitted. All root systems were stained with 0.15 g/L phloxine B for 15 min to visualize M. chitwoodi egg masses as well as facilitate counting under a Stemi 2000C stereomicroscope (Zeiss). At 55 DAI root systems were washed free of substrate and for potato, root fresh weights were determined as before. For each root system, eggs were extracted as described above and eggs were counted under a stereomicroscope. Eggs were used to extract RNA or hatch infective J2 for qRT-PCR assays as detailed above. All infection assays were conducted twice and showed similar results. Representative results are shown.

### Northern Blots for Transgenic Arabidopsis and Potato Lines

Total RNA and small RNA were extracted from 0.5 g leaf tissue of each line using the MIRVANA™ miRNA isolation kit (Ambion, Austin, Tex., USA). For each line, total RNA (20 μg for Arabidopsis and 4 μg for potato) was denatured in 50% deionized formamide (Amresco, Solon, Ohio, USA), separated on a 1% agarose gel in 1×TBE buffer (0.9 M Tris base, 0.9 M boric acid, 20 nM EDTA), and transferred by capillary action onto NYTRAN™ N nylon membrane (Sigma, St. Louis, Mo., USA) in 10×SSC (1.5 M NaCl, 0.15 M sodium citrate, pH 7.0). In addition, 1 μg denatured small RNA was separated on a 15% polyacrylamide gel with 8 M urea in 1×TBE buffer, and transferred onto NYTRAN™ N membrane as described above. For northern blotting, [α-³²P] dATP probes (MP Biochemicals, Solon, Ohio, USA) Mi16D10 and U6 were synthesized by PCR using primers 16D10F5, 16D 10R5, U6F and U6R, respectively (Table 1). Probes were labeled radioactively and purified with DECAPRIME™ II kit (Ambion) and Illustra NICK columns (GE Healthcare Life Sciences, Pittsburgh, Pa., USA). Membranes were UV-crosslinked and hybridized with radioactive probes overnight at 37° C. in hybridization buffer (50% deionized formamide, 3×SSC, 0.1 mg/mL salmon sperm DNA, 1% sodium dodecyl sulfate, 0.05 M phosphate buffer, 0.2% bovine serum albumin, 0.2% polyvinylpyrrolidone, 0.2% Ficoll) before being washed three times in 2×SSC and 0.2% SDS for 20 min at 46° C. and exposed to X-ray films for 1-5 days at −80° C. All experiments were conducted twice and showed similar results.

TABLE 1
Primers and probes used for cloning, PCR, northern and Southern blots.

| Gene | Primer name | Primer sequences (5′-3′) |
|---|---|---|
| Mi16D10 | 16D10F5 | GTTTACTAATTCAATTAAAAATTTAATT (SEQ ID NO: 8) |
| (DQ087264) | 16D10R5 | CAATTATTTCCTCCAGGATTTGGCCC (SEQ ID NO: 9) |
| U6 | U6F | GCGCAAGGATGACACGCA (SEQ ID NO: 10) |
| (X60506) | U6R | GGCTGAGTTATTTTTTTCTG (SEQ ID NO: 11) |
| ITS2 | McITS-RTF | GGGGTCAAACCCTTTGGCACGTCTGG (SEQ ID NO: 12) |
| (JN241865) | McITS-RTR | GCGGGTGATCTCGACTGAGTTCAGG (SEQ ID NO: 13) |
| Mc16D1OL | Mc16D10-F | GATATTTAAATTAAATTATATTCTTCTAAA (SEQ ID NO: 14) |
| (CD418743) | Mc16D10-R | GCTTTATTCAATTTATTTTTATTTATT (SEQ ID NO: 15) |
| | Mc16D10-RTF | TTATTTTATCTGTTACTTTTGTGGATTCAGC (SEQ ID NO: 16) |
| | Mc16D10-RTR | GCGACCATCATTATTATCATTTCCACC (SEQ ID NO: 17) |
| 16D10dsRNA | 35S-F | TTCGCAAGACCCTTCCTCTA (SEQ ID NO: 18) |
| | OCS1 | CTTCTTCGTCTTACACATCACTTGTC (SEQ ID NO: 19) |

### Quantitative Real-Time PCR to Study Transcript Levels of Mc16D10L

To analyze the transcript levels of Mc16D10L throughout the life cycle of M. chitwoodi, qRT-PCR was performed with cDNA from different nematode life stages harvested from greenhouse nematode stock cultures, as well as from eggs and infective J2 originating from plants used in infection assays (see above). Total RNA was extracted and cDNA generated as described above. qRT-PCR was performed in an iQ Real-Time PCR machine (Bio-Rad, Hercules, Calif., USA) using the IQ™ SYBR® Green Supermix kit (Bio-Rad). Internal transcribed spacer 2 (ITS2) rRNA (JN241865) was chosen as internal control gene for qRT-PCR after comparing the expression pattern with that of other M. chitwoodi housekeeping genes, such as 18S rRNA (AY757835), actin 2 (CB930959) and glyceraldehyde-3-phosphate-dehydrogenase-1 (CB9332930). Primers McITS-RTF and McITS-RTR were designed based on the M. chitwoodi ITS2 gene, and used for internal control qRT-PCR reactions (Table 1). Mc16D10L was amplified using primers Mc16D10-RTF and Mc16D10-RTR (Table 1). Each sample was run in triplicate using the following program: 94° C. for 10 min; 45 cycles of 94° C. for 30 s, 50° C. for 30 s and 72° C. for 30 s, followed by 91 cycles with a temperature increase of 0.5° C. after each cycle from 50° C. to 95° C. The differences in the expression level of Mc16D10L in M. chitwoodi were analyzed using the 2^{-ΔΔCt} method (Livak and Schmittgen, 2001) with Ct values exported from iQ5 Optical System Software (Bio-Rad). All experiments were conducted at least twice and showed similar results. Representative results are shown.

### Data Analysis

Number of egg masses, number of eggs, fold changes and log₁₀ fold changes of Mc16D10L expression were analyzed in Microsoft Excel to calculate means and standard errors. Statistically significant differences were estimated using a Student's t-test with alpha=0.05 in SAS 9.2.

### Results

M. chitwoodi Effector Gene Mc16D10L is a Homolog of Mi16D10

In order to identify a putative M. chitwoodi homolog of the M. incognita effector gene Mi16D10, M. chitwoodi expressed sequence tags (ESTs) were searched using BLASTN (Huang et al., 2006b; Roze et al., 2008). M. chitwoodi EST CD418743 provided the best match and a full-length sequence spanning the complete open reading frame was cloned from infective J2 cDNA using the gene-specific primers Mc16D10-F and Mc16D10-R (Table 1). The coding sequence of this Mi16D10-like M. chitwoodi homolog, which will be referred to as Mc16D10L henceforth, had a length of 153 bp. A pairwise sequence alignment showed that 70% of the sequences of Mi16D10 and Mc16D10L were identical on the nucleotide level and 63% on the amino acid level, respectively (FIG. 1). The N-terminal 32 amino acids of Mc16D10L represent a signal peptide, a characteristic of nematode effector gene products and indicating that this putative effector peptide is most likely secreted. Importantly, a region with similarity to the plant CLAVATA3 (CLV3)/ENDOSPERM SURROUNDING REGION (ESR) (CLE) motif (KRXVPXGPNPLHNR, SEQ ID NO: 19) found in Mi16D10 and other Meloidogyne spp. 16D10 orthologs also was conserved in Mc16D10L. The nucleotide sequence of Mc16D10L was deposited in GenBank under accession number KF734590.

Mc16D10L Expression Changes during Nematode Development

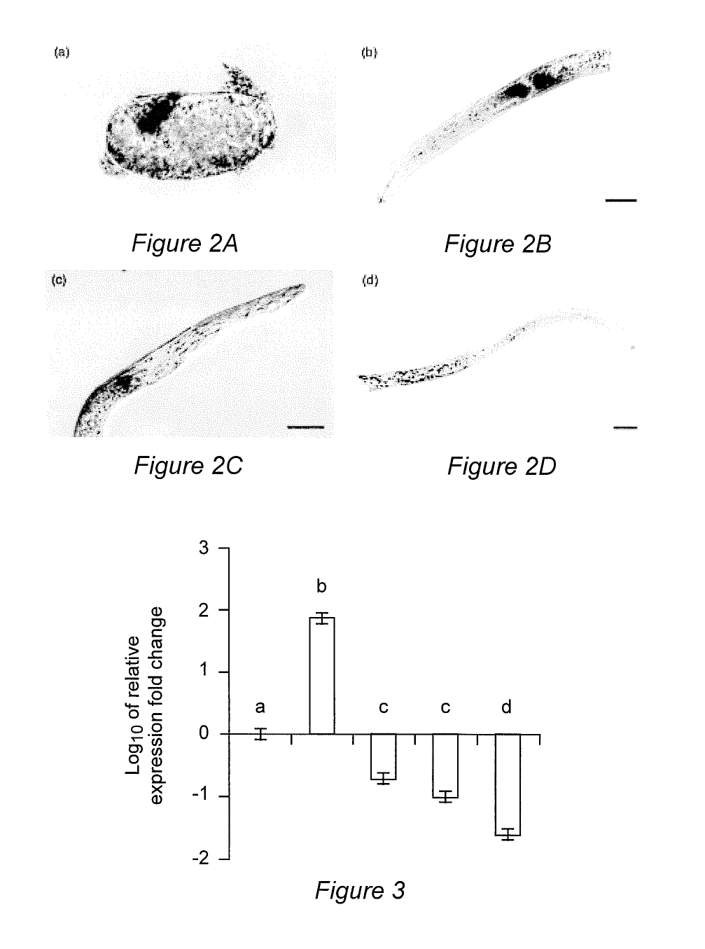

In situ hybridizations showed that Mc16D10L was expressed most strongly in the subventral gland cells of M. chitwoodi infective J2 (FIG. 2). Weaker expression was found in eggs and subventral gland cells of parasitic J2, whereas no gene activity could be detected in late parasitic life stages by in situ hybridizations. To corroborate the in situ data, quantitative real-time PCR (qRT-PCR) was performed with cDNA from all M. chitwoodi life stages except adult males (FIG. 3). Using the expression level of Mc16D10L in eggs as reference, Mc16D10L was upregulated 1.87-fold in infective J2 on a log₁₀ scale, which represents a 78-fold upregulation in absolute terms. In parasitic J2, mixed J3/J4 and adult females, Mc16D10L was expressed at a lower level than in eggs, with a fold change of −0.72, −1 and −1.6 on a log₁₀ scale (5-fold, 10-fold and 33-fold downregulation in absolute terms), respectively.

Plant-Mediated RNAi of Mc16D10L Increases M. chitwoodi Resistance

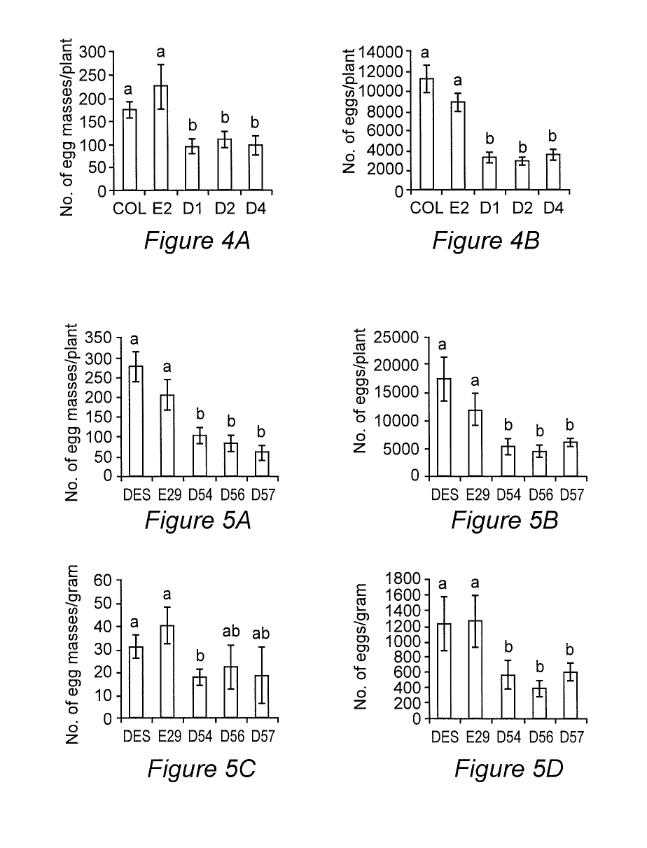

To test whether in planta expression of dsRNA that is complementary to Mc16D10L provides resistance against M. chitwoodi, three T₃ Arabidopsis lines carrying the RNAi construct pART27(16D10i-2) were inoculated with 5,000 M. chitwoodi eggs per plant. No overt phenotypical changes were observed in transgenic plants compared to wild type controls. At 35 days after inoculation (DAI), the average number of egg masses per plant was reduced significantly (P<0.05) by 36-45% in RNAi lines D1, D2 and D4 compared to the Col-0 wild type control, and by 50-57% compared to the empty vector control, respectively. At 55 DAI, the average number of eggs per plant in Arabidopsis RNAi lines was reduced significantly by 68-74% compared to the wild type control, and by 59-67% relative to the empty vector control (FIG. 4). Since expression of dsRNA of Mc16D10L led to enhanced resistance against M. chitwoodi in the model plant Arabidopsis, the RNAi construct pART27(16D10i-2) was used to generate stable transgenic potato lines to test whether the same strategy could be applied to engineer M. chitwoodi resistance in potato. Three transgenic RNAi potato lines (D54, D56, D57), an empty vector and a wild type control line were inoculated with 5,000 M. chitwoodi eggs per plant. No overt phenotypical changes were observed in transgenic potato plants compared to wild type controls. At 35 DAI the average number of egg masses per plant was reduced significantly by 63-79% compared to the wild type control, and by 50-71% compared to the empty vector control (P<0.05). Similarly, the average number of eggs per RNAi plant at 55 DAI was reduced significantly by 65-74% compared to the wild type, and by 50-63% compared to the empty vector control lines (P<0.05). Comparable results were obtained when nematode infection was measured as number of egg masses or number of eggs per gram root fresh weight in potato, with significant reductions of 44-56% and 53-69% (P<0.05) compared to the empty vector control, respectively (FIG. 5). Northern blots confirmed the expression of small RNAs ranging in size from about 50 to over 150 nt in all transgenic pART27(16D10i-2) Arabidopsis and potato lines when a 16D10 probe was used (FIG. 6).

Potato RNAi Lines Downregulate Mc16D10L in M. chitwoodi

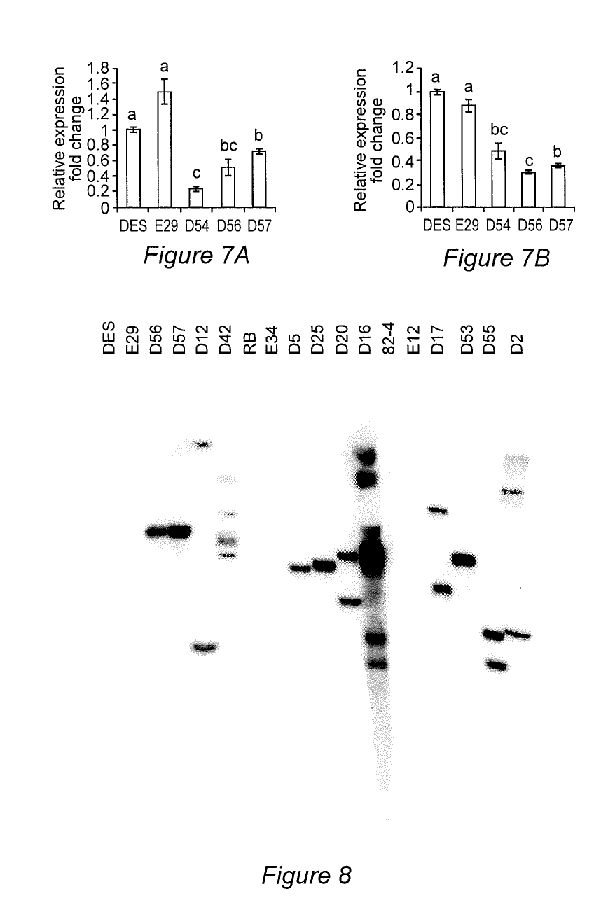

To investigate the effect of plant-mediated RNAi on the activity of the target gene in nematodes, qRT-PCR was used to analyze the relative transcript level of Mc16D10L in M. chitwoodi eggs and J2 harvested from potato plants at 55 DAI. Using the internal ribosomal spacer 2 (ITS2) of nuclear ribosomal DNA as reference (McClure et al., 2012), it was found that the expression level of Mc16D10L in M. chitwoodi eggs that developed on potato plants carrying pART27(16D10i-2) was reduced significantly by on average 27-76% compared to eggs harvested from wild type control plants (P<0.05). Similarly, the expression level of Mc16D10L in infective J2 that hatched from eggs harvested from potato RNAi lines was reduced significantly (P<0.05) by 52-70% relative to J2 that developed from eggs collected from wild type control plants (FIG. 7).

### Discussion

Root-knot nematodes infect virtually all vascular plants and are a major problem in a wide variety of crops. This study demonstrates that plant-mediated downregulation of the putative effector gene Mc16D10L provides resistance against M. chitwoodi in stable transgenic lines of Arabidopsis and potato. To the best of our knowledge, this is the first report of stable transgenic RNAi potato lines with resistance against nematodes.

Stable transgenic Arabidopsis and potato lines overexpressing a 271 nt full-length 16D10 dsRNA construct showed strong resistance against M. chitwoodi in this study. Compared to the empty vector control, the number of egg masses at 35 DAI was reduced significantly by 50-57% in Arabidopsis and by 50-71% in potato, respectively (P<0.05). Similarly, the number of eggs formed by 55 DAI was significantly reduced in both Arabidopsis and potato lines compared to the empty vector control, with 59-67% and 50-63%, respectively (P<0.05). No overt changes in plant morphology were observed in the Arabidopsis and potato lines generated in the experiments described here and the level and range of resistance is comparable with what has been reported in other plant-mediated RNAi systems in which cyst or root-knot nematode genes have been targeted.

Importantly in this study, qRT-PCR indicated a significant reduction in Mc16D10L transcripts in the second-generation M. chitwoodi eggs and infective J2 that developed on the transgenic pART27(16D10i-2) potato lines. We hypothesize that the RNAi effect of Mc16D10L in the nematode is systemic and proliferates upon the initial uptake of plant-derived dsRNAs or siRNAs from the esophagus through the entire body of the female, including the gonads and developing eggs, thereby transmitting the RNAi effect to the offspring.

In spite of repeated attempts it was not possible to verify the production of 16D10-specific siRNAs in the Arabidopsis and potato lines created here, a problem that has been encountered previously for other genes, and most likely due to the limited sensitivity of northern blot assays (Charlton et al., 2010; Zilberman et al., 2003). Meloidogyne spp. generate a feeding tube during each feeding cycle and it may act as a filter to prevent clogging of the nematode's mouth spear (Hussey and Mims, 1991). Earlier studies have shown that root-knot nematodes are able to ingest molecules of 28-140 kDa, including the green fluorescent protein and crystal proteins formed by the biocontrol agent Bacillus thuringiensis. It is unknown if M. chitwoodi took up plant-derived dsRNAs and processed them into siRNAs or whether the nematode directly ingested plant-produced siRNAs in this study. Given that Meloidogyne spp. are able to ingest relatively large molecules, either possibility is conceivable. Previous experiments have demonstrated that a match of 21 nt or less between siRNAs and a target sequence is sufficient to trigger RNAi in animals and that several mismatched basepairs do not interfere with the silencing process (Hutvágner et al., 2001; Jackson et al., 2003; Parrish et al., 2000). There are numerous conserved regions between Mi16D10 (upon which pART27(16D10i-2) was designed) and Mc16D10L that fall within this size range and could thereby trigger RNAi in M. chitwoodi.

Almost all commercial potato cultivars are autotetraploid (2n=4x=48), which makes classic breeding schemes complicated and time-consuming, especially if the high level of heterozygosity related to tetrasomic inheritance is taken into consideration. Introgressing traits from wild Solanum spp., e.g. nematode resistance genes, compound already challenging breeding strategies and have to overcome additional complications, such as pre- and postzygotic incompatibility barriers. Root-knot nematode resistance in wild Solanum spp. is relatively poorly characterized and a largely untapped resource. Given the challenges associated with classic potato breeding, transgenic strategies as described here present an attractive alternative to breeding root-knot nematode-resistant potatoes. One of the advantages of creating transgenic plants with an RNAi-based resistance against nematodes is that in principle no foreign protein is expressed in planta, thereby making the end product potentially more desirable than transgenic crops that express nematicidal peptides or proteins, such as cystatins or neurotransmitter antagonists. Ideal candidate genes for RNAi-based approaches are specific and only present in the target organisms, even though it has been reported that silencing of host genes can also result in decreased susceptibility against cyst and root-knot nematodes in Arabidopsis. Meloidogyne effectors usually lack homology to genes in other taxa, which makes them a worthwhile group of target genes for safe and specific root-knot nematode control. Furthermore, RNAi constructs against more than one effector gene can be stacked to achieve additive effects and aid in creating a more durable nematode resistance (Charlton et al., 2010). In summary, the experiments described here demonstrate that specific silencing of the putative effector gene Mc16D10L leads to M. chitwoodi resistance not only in Arabidopsis but also in stable transgenic lines of potato, thereby opening the door to improved molecular breeding strategies for nematode resistance in this extremely important food crop.

### References for Example 1

Brown, C. R., Mojtahedi, H. and Santo, G. S. (1991a) Resistance to Columbia root-knot nematode in Solanum spp. and in hybrids of S. hougasii with tetraploid cultivated potato. Amer. Potato J., 68, 445-452.
Brown, C. R., Yang, C. P., Kwiatkowski, S. and Adiwilaga, K. D. (1991b) Insert copy number, chromosome-number, pollen stainability, and crossability of Agrobacterium-transformed diploid potato. Amer. Potato J., 68, 317-330.
Charlton, W. L., Harel, H. Y. M., Bakhetia, M., Hibbard, J. K., Atkinson, H. J. and McPherson, M. J. (2010) Additive effects of plant expressed double-stranded RNAs on root-knot nematode development. Int. J. Parasitol., 40, 855-864.
Clough, S. J. and Bent, A. F. (1998) Floral dip: A simplified method for Agrobacterium-mediated transformation of Arabidopsis thaliana. Plant J., 16, 735-743.
Dinh, P. T. Y., Knoblauch, M. and Elling, A. A. (2014) Non-destructive imaging of plant-parasitic nematode development and host response to nematode pathogenesis. Phytopathology, in press (dx.doi.org/10.1094/PHYTO-08-13-0240-R).
Huang, G., Allen, R., Davis, E. L., Baum T. J. and Hussey, R. S. (2006a) Engineering broad root-knot resistance in transgenic plants by RNAi silencing of a conserved and essential root-knot nematode parasitism gene. Proc. Natl. Acad. Sci. USA, 103, 14302-14306.
Huang, G., Dong, R., Allen, R., Davis, E. L., Baum, T. J. and Hussey, R. S. (2006b) A root-knot nematode secretory peptide functions as a ligand for a plant transcription factor. Mol. Plant-Microbe Interact., 19, 463-470.
Humphreys-Pereira, D. A. and Elling, A. A. (2013) Intraspecific variability and genetic structure in Meloidogyne chitwoodi from the USA. Nematology, 15, 315-327.
Humphreys-Pereira, D. A. and Elling, A. A. (2014) Morphological variability in second-stage juveniles and males of Meloidogyne chitwoodi. Nematology, 16, in press (doi:10.1163/15685411-00002753).
Hussey, R. S. and Barker, K. R. (1973) A comparison of methods of collecting inocula of Meloidogyne spp., including a new technique. Plant Dis. Rep., 57, 1025-1028.
Hussey, R. S. and Mims, C. W. (1991) Ultrastructure of feeding tubes formed in giant-cells induced in plants by the root-knot nematode Meloidogyne incognita. Protoplasma, 162, 99-107.
Hussey, R. S., Huang, G. and Allen, R. (2011) Microaspiration of esophageal gland cells and cDNA library construction for identifying parasitism genes of plant-parasitic nematodes. Methods Mol. Biol., 712, 89-107.
Hutvágner, G., McLachlan, J., Pasquinelli, A. E., Bálint, E., Tuschl, T. and Zamore, P. D. (2001) A cellular function for the RNA-interference enzyme Dicer in the maturation of the let-7 small temporal RNA. Science, 293, 834-838.
Jackson, A. L., Bartz, S. R., Schelter, J., Kobayashi, S. V., Burchard, J., Mao, M., Li, B., Cavet, G. and Linsley, P. S. (2003) Expression profiling reveals off-target gene regulation by RNAi. Nat. Biotechnol., 21, 635-637.
Livak, K. J. and Schmittgen, T. D. (2001) Analysis of relative gene expression data using real-time quantitative PCR and the 2^{-ΔΔCt} method. Methods, 25, 402-408.
McClure, M. A., Nischwitz, C., Skantar, A., Schmitt, M. E. and Subbotin, S. A. (2012)

Root-knot nematodes in golf course greens of the western United States. Plant Dis., 96, 635-647.
Parrish, S., Fleenor, J., Xu, S., Mello, C. and Fire, A. (2000) Functional anatomy of a dsRNA trigger: Differential requirement for the two trigger strands in RNA interference. Mol. Cell, 6, 1077-1087.
Roze, E., Hanse, B., Mitreva, M., Vanholme, B., Bakker, J. and Smant, G. (2008) Mining the secretome of the root-knot nematode Meloidogyne chitwoodi for candidate parasitism genes. Mol. Plant Pathol., 9, 1-10.
Sheerman, S. and Bevan, M. W. (1988) A rapid transformation method for Solanum tuberosum using binary Agrobacterium tumefaciens vectors. Plant Cell Rep., 7, 13-16.
Zilberman, D., Cao, X. and Jacobsen, S. E. (2003) ARGONAUTE4 control of locus-specific siRNA accumulation and DNA histone methylation. Science, 299, 716-719.

### Example 2

Plant-mediated RNA interference of effector gene Mc16D10L confers resistance against Meloidogyne chitwoodi in diverse genetic backgrounds of potato and reduces pathogenicity of nematode offspring

Summary—In this study, an RNA interference (RNAi) transgene targeting the M. chitwoodi effector gene Mc16D10L was introduced into potato cvs. Russet Burbank and Désirée, and the advanced breeding line PA99N82-4, which carries the R_{Mc1} gene. Stable transgenic lines were generated for greenhouse infection assays. At 35 days after inoculation (DAI) with M, chitwoodi, the number of egg masses per gram root formed on RNAi lines of cvs. Russet Burbank and Désirée was reduced significantly by up to 68% compared to empty vector control plants. At 55 DAI, the number of eggs was reduced significantly by up to 65%. In addition, RNAi of Mc16D10L significantly reduced the development of egg masses and eggs formed by the R_{Mc1} resistance-breaking M. chitwoodi pathotype Roza on PA99N82-4 by up to 47 and 44%, respectively. Importantly, the plant-mediated silencing effect of Mc16D10L was transmitted to M. chitwoodi offspring and significantly reduced pathogenicity in the absence of selection pressure in empty vector control plants. This finding suggests that the RNAi effect is stable and nematode infection decreases regardless of the genotype of the host once the RNAi process has been initiated through a transgenic plant. In summary, plant-mediated downregulation of effector gene Mc16D10L provides a promising new tool for molecular breeding against M. chitwoodi.

Accordingly, in this report, RNAi technology was used to develop M. chitwoodi resistance in potato cvs. Russet Burbank and Désirée, and advanced breeding line PA99N82-4. Russet Burbank makes up about 40% of the total potato acreage in the USA, and Désirée is an important specialty cultivar, particularly in Europe. PA99N82-4 carries the R_{Mc1} resistance gene from S. bulbocastanum (Brown et al., 2009). The objectives of this study were to (i) analyse whether the genetic background of potato has an effect on 16D10-RNAi-mediated M. chitwoodi resistance, (ii) test whether 16D10-RNAi resistance is effective against the R_{Mc1}-breaking M. chitwoodi pathotype Roza, and (iii) determine whether introduction of 16D 10-RNAi reduces the pathogenicity of M. chitwoodi offspring to potato.

### Materials and Methods

### Generation of Transgenic Potato Lines

Internodal stem segments of potato cvs. Désirée and Russet Burbank, as well as advanced breeding line PA99N82-4 were co-incubated with Agrobacterium tumefaciens strain GV3101 carrying the RNAi silencing construct pART27(16D10i-2) or empty vector control pART27 (Brown et al., 2006; Huang et al., 2006a). After three days on CIM media (MS basal salts, 0.25 ppm folic acid, 0.05 ppm D-biotin, 2 ppm glycine, 0.5 ppm nicotinic acid, 0.5 ppm pyridoxine HCl, 0.4 ppm thiamine HCl, 0.01% myo-inositol, 3% D-sucrose, 1 ppm 6-benzylaminopurine, 2 ppm 1-naphthaleneacetic acid, 0.6% Daishin agar at pH 5.6), stem segments were transferred to 3C5ZR media (MS basal salts, 0.5 ppm nicotinic acid, 0.5 ppm pyridoxine HCl, 1 ppm thiamine HCl, 0.01% myo-inositol, 3% D-sucrose, 0.5 ppm indole-3-acetic acid, 3 ppm zeatin ribose, 0.5 g/l timentin, 70 μg/ml kanamycin sulfate, 0.6% Daishin agar at pH 5.9), and incubated in a growth chamber (22° C., 12 h photoperiod) for about three months until shoots developed (Sheerman & Bevan, 1988; Brown et al., 1991b; and see Example 1). Stem segments were transferred to fresh 3C5ZR media every two weeks. Potato plantlets that regenerated on 3C5ZR were maintained on propagation media (MS basal salts, supplemented with 3% D-sucrose, 50 μg/ml kanamycin sulfate and 50 μg/ml timentin, solidified with 0.6% Daishin agar) in a growth chamber at 22° C. with a 12 h photoperiod (and see Example 1). Wild type lines of cv. Désirée and Russet Burbank and PA99N82-4 also were treated the same way, except that the propagation media did not contain kanamycin sulfate and timentin.

### DNA Extraction and Southern Blotting of Transgenic Potato Lines

DNA was extracted from leaves of all putative pART27(16D10i-2) and pART27 transformants that survived kanamycin selection. DNA extracted from wild type cvs. Désirée and Russet Burbank and advanced breeding line PA99N82-4 served as controls. Approximately 1 g leaf material was ground in liquid nitrogen and homogenised in 3 ml TPS extraction buffer (100 mM Tris-HCl (pH 8.0), 100 mM EDTA (pH 8.0), 1 M KCl). The resulting leaf extraction suspensions were transferred to 15 ml tubes, incubated at 75° C. for 10 min, and centrifuged at 13500×g for 10 min. Supernatants were collected and mixed with an equal volume of isopropanol by inverting each tube several times. DNA was pelleted by centrifuging at 2500×g for 10 min, washed with 70% ethanol, air dried, resuspended in 0.4 ml sterile water containing 0.5 mg/ml RNase (Fermentas) and incubated at 37° C. for 30 min After RNase treatment, DNA samples were mixed with 0.4 ml chloroform, vigorously shaken for 1 min and centrifuged at 13500×g for 5 min After centrifugation, the top layer of each tube was collected and mixed with an equal volume of isopropanol by inverting each tube several times. Samples were centrifuged at 18000×g for 10 min, and pelleted DNA was washed with 70% ethanol, air dried and resuspended in 100 μl sterile water.

Prior to Southern blotting, DNA from putative transformants was analysed by PCR for presence of the transgene as described in Example 1, using primers 35S-F and OCS1 for pART27(16D10i-2) and primers nptII-F and nptII-R for pART27, respectively (data not shown). For Southern blots, 15 μg DNA of each PCR-positive potato line was digested with 50 U XbaI (New England Biolabs) for 16 h at 37° C. Digested DNA was separated on a 0.8% agarose gel at 70 V for 16 h before being transferred by capillary action to a GENESCREEN PLUS® nylon membrane (PerkinElmer) in 10× saline sodium citrate (SSC) buffer (1.5 M NaCl, 0.15 M sodium citrate, pH 7.0). The membranes were cross-linked by UV, and hybridised overnight in hybridisation buffer (50% deionised formamide, 0.1 mg/ml salmon sperm DNA, 1% sodium dodecyl sulfate (SDS), 1 M NaCl, 10% dextran sulfate) at 42° C. with probe 16D10i-2. The probe was amplified with primers 35S-F and OCS1 using plasmid pART27(16D10i-2) as template, radioactively labeled with [α-³²P] dATP (MP Biochemicals) using the DECAPRIME™ II kit (Ambion) and purified with Illustra NICK columns (GE Healthcare Life Sciences). After hybridisation, the membranes were washed twice with 2×SSC buffer for 5 min at 42° C., followed by three washes with 2×SSC plus 1% SDS for 20 min at 65° C. Three final washes were carried out with 0.1×SSC plus 1% SDS for 20 min each at 42° C., after which the membranes were exposed to X-ray films (Research Products International) for 2 days at −80° C. All experiments were conducted twice, and showed similar results.

### Northern Blotting of Transgenic Potato Lines

Total RNA enriched with small RNAs was extracted from 1 g of leaves using the MIRVANA™ miRNA isolation kit (Ambion). For each potato line, 20 μg denatured total RNA was separated on a 1% agarose gel at 120 V for 1 h before being transferred by capillary action to NYTRAN™ N nylon membranes (Sigma-Aldrich) overnight. Probes 16D10 and U6 were synthesized using primers 16D10F5, 16D10R5, U6F and U6R (Table 1 of Example 1 and the additional primers shown in Table 2 below), respectively, with plasmid pART27(16D10i-2) and potato cDNA serving as templates. Probes were radioactively labeled with [α-³²P] dATP (MP Biochemicals) as described above, and used to hybridize the membranes overnight at 25° C. in hybridization buffer (50% deionised formamide, 3×SSC, 0.1 mg/ml salmon sperm DNA, 1% SDS, 0.05 M phosphate buffer, 0.2% bovine serum albumin, 0.2% polyvinylpyrrolidone, 0.2% Ficoll). After hybridisation, membranes were washed three times with 2×SSC plus 0.2% SDS for 20 min at 46° C. before being exposed to X-ray films (Research Products International) for 1-7 days at −80° C. All experiments were conducted at least three times, and showed similar results.

TABLE 2
Primers and probes used for PCR, Southern and northern blots.

| Probe/gene | Primer name | Primer sequences (5′-3′) |
|---|---|---|
| 16D10-i2 | nptII-F | ATCGGGAGCGGCGATACCGTA (SEQ ID NO: 20) |
| | nptII-R | GACGCTATTCGGCTATGACTG (SEQ ID NO: 21) |

### Nematode Inoculum and Infection Assays

M. chitwoodi isolates WAMC1 (race 1) and Roza (race 1, pathotype Roza) were maintained on tomato (S. lycopersicum) cv. Rutgers under greenhouse conditions. To ensure isolate purity, a portion of each batch of inoculum was used in as assay with indicator host plants as described previously (Brown et al., 2009; Humphreys-Pereira & Elling, 2013). To obtain nematode inoculum, M. chitwoodi eggs were collected from tomato plants that were inoculated about three months earlier following routine procedures (Hussey & Barker, 1973). Briefly, infected roots were cut into 2-3 cm pieces and shaken in 0.5% NaOCl for 3 min. The root suspension was poured over a set of nested test sieves (850, 75, 25 μm pore size from top to bottom) and eggs were collected on the 25 μm pore sieve. For nematode offspring RNAi infection assays (see below), M. chitwoodi WAMC1 eggs were collected from potato cv. Désirée lines E29 (carrying empty vector pART27) and D56 (carrying RNAi silencing construct pART27(16D10i-2)) that were inoculated with M. chitwoodi eggs four months earlier and maintained under greenhouse conditions. Egg extractions were the same as described above.

Transgenic pART27(16D10i-2) potato lines, with cv. Désirée (D56, D57, D12 and D42), cv. Russet Burbank (D5, D16, D20 and D25) and PA99N82-4 (D2, D17, D53 and D55) as genetic backgrounds, were chosen for infection assays based on transgene copy numbers and overall phenotypic appearance. Single nodes were cut from each line and maintained for 1 month on propagation media supplemented with 50 μg/ml kanamycin sulfate. Similarly, single nodes of wild type and pART27 empty vector control lines of each genetic background (DES and E29 for cv. Désirée, RB and E34 for cv. Russet Burbank, 82-4 and E12 for PA99N82-4, respectively) were cut and maintained on propagation media lacking kanamycin sulfate for an equal amount of time. One month-old plantlets were transferred to individual Ray Leach SC10U cone-tainers (Stuewe & Sons) filled with autoclaved sand. Cone-tainers were placed in RL98 trays (Stuewe & Sons) and plants were allowed to acclimate to greenhouse conditions for 10 days, after which each cone-tainer was inoculated with 2000 M. chitwoodi eggs. Plants were maintained in the greenhouse for the duration of the experiment, and watered twice a day with 20-10-20 NPK liquid fertiliser. Infection assays were set up as randomized complete block designs with 10 replicates (10 plants) per line and timepoint (35 and 55 days after inoculation, DAI). All experiments were conducted twice, and showed similar results. Three distinct plant/nematode combinations were analyzed: (i) potato cv. Désirée, cv. Russet Burbank and PA99N82-4 inoculated with M. chitwoodi isolate WAMC1, (ii) PA99N82-4 inoculated with M. chitwoodi isolate Roza, and (iii) cv. Désirée inoculated with M. chitwoodi isolate WAMC1 collected from potato lines with and without the 16D10i-2 transgene. For (i) and (ii), nematode inoculum was collected from tomato cv. Rutgers as described above and the infection assays were conducted in the greenhouse. For (iii), which was designed to test the RNAi effect on nematode offspring, M. chitwoodi inoculum was harvested from transgenic potato cv. Désirée lines E29 (empty vector control) and D56 (carrying pART27(16D10i-2)), resulting in four different treatments: empty vector line E29 inoculated with M. chitwoodi eggs collected from line E29 (pE29-eE29); empty vector line E29 inoculated with M. chitwoodi eggs collected from 16D10i-2 line D56 (pE29-eD56); 16D10i-2 line D56 inoculated with M. chitwoodi eggs collected from empty vector line E29 (pD56-eE29); and 16D10i-2 line D56 inoculated with M. chitwoodi eggs collected from line D56 (pD56-eD56), where ‘p’ stands for plant and ‘e’ stands for eggs. pE29 and pD56 served as additional controls, and were inoculated with M. chitwoodi WAMC1 eggs collected from wild type tomatoes cv. Rutgers. Eggs harvested from pE29 and pD56 served as inoculum for the infection assays in (iii). Nematode infection assays for (iii) were conducted in a growth chamber (25° C. day, 21° C. night, 16 h photoperiod).

For each plant/nematode combination and experiment, 10 plants were harvested at 35 DAI, their roots were washed free of sand and the fresh weight of roots was determined. Root systems were stained with 0.15 g/l phloxine B for 15 min to visualise egg masses, and facilitate counting under a stereomicroscope. At 55 DAI, an additional 10 plants were harvested, the roots washed, and root fresh weight determined as before. Eggs were extracted as described above, and again counted under a stereomicroscope.

### Nematode RNA Extraction, Quantitative Real-Time PCR and Northern Blots

For infection assay (iii) (see above) an aliquot of each batch of eggs harvested from experimental plants at 55 DAI was used to hatch J2 in a modified Baermann pan following established procedures (see Example 1). Second-stage juveniles were collected by centrifugation and flash frozen in liquid nitrogen. Total RNA was extracted from J2 with the PerfectPure RNA Fibrous Tissue kit (SPrime). For qRT-PCR, 500 ng total RNA was used to synthesize a total volume of 100 μl cDNA with the ADVANTAGE® RT-for-PCR kit (Clontech). qRT-PCR for analysing the transcript level of Mc16D10L was conducted in an iQ Real-Time PCR machine with IQ™ SYBR® Green Supermix (Bio-Rad) (see Example 1). Primers Mc16D10-RTF and Mc16D10-RTR were used to amplify target gene Mc16D10L. Internal transcribed spacer 2 (ITS2) rRNA (JN241865) served as the control, and was amplified with primers McITS-RTF and McITS-RTR (Table 1) (see Example 1). Differences in transcript levels were analysed using the 2^{-ΔΔCt} method (Livak & Schmittgen, 2001) with Ct values retrieved from IQ™ 5 Optical System Software (Bio-Rad). All reactions were run in triplicates, conducted twice and gave similar results.

For northern blots, RNA was transferred to NYTRAN™ N membranes (Sigma-Aldrich) and hybridized with probe Mc16D10L (Table 1), generated with primers Mc16D10-F and Mc16D10-R, and radioactively labeled with [α-³²P] dATP (MP Biochemicals) as described above. As the control, a probe for ITS2 was generated with primers McITS-RTF and McITS-RTR (Table 1).

### Data Analysis

Number of egg masses, number of eggs and relative fold changes of Mc16D10L transcript levels were analyzed in Microsoft Excel to calculate means and standard errors. Statistically significant differences were estimated in SAS 9.2 using a Student's t-test with alpha=0.05.

### Results

RNAI Transgene 16D101-2 Increases Resistance Against M. chitwoodi in Different Genetic Backgrounds of Potato

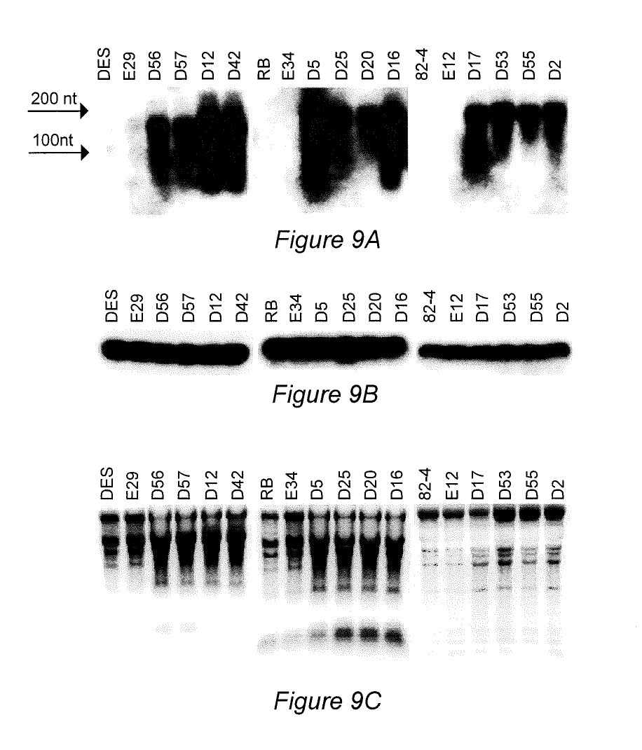

Stable transgenic lines of cv. Désirée, cv. Russet Burbank and PA99N82-4 each carrying the silencing construct pART27(16D10i-2) were generated to test whether the genetic background of potato has an effect on plant-mediated RNAi resistance against M. chitwoodi. Empty vector lines transformed with pART27 and wild type plants served as controls. No overt phenotypical changes were observed in transgenic plants compared to wild type controls throughout development. To examine whether the copy number of the RNAi transgene is related to the level of nematode resistance, transgenic potato lines were analyzed by Southern blotting and representative lines with single, double and multiple insertions were chosen from each genetic background for subsequent experiments (FIG. 8). Lines D56 and D57 of cv. Désirée had single insertions of 16D10i-2, D12 had a double insertion, and D42 showed multiple copies. Similarly, cv. Russet Burbank lines D5 and D25 had single, D20 had double and D16 had multiple insertions of the RNAi transgene. PA99N82-4 line D53 carried a single copy of 16D101-2, D17 and D55 had two insertions, and D2 had multiple insertions. Northern blots indicated that a greater number of pART27(16D10i-2) transgene insertions does not necessarily lead to an increased level of 16D101-2-specific small RNAs (FIG. 2). Even though the expression level of 16D101-2 small RNAs was higher in cv. Désirée lines D12 and D42 (having double and multiple insertions of the RNAi transgene, respectively) than in single insertion lines D56 and D57, such relationships were not found among some of the other lines. For example, cv. Russet Burbank line D5 only had a single insertion of pART27(16D10i-2), but its 16D10i-2 small RNA level was considerably higher than that of other lines of the same genetic background with single, double and multiple insertions. Similar results were obtained in PA99N82-4 (FIG. 9).

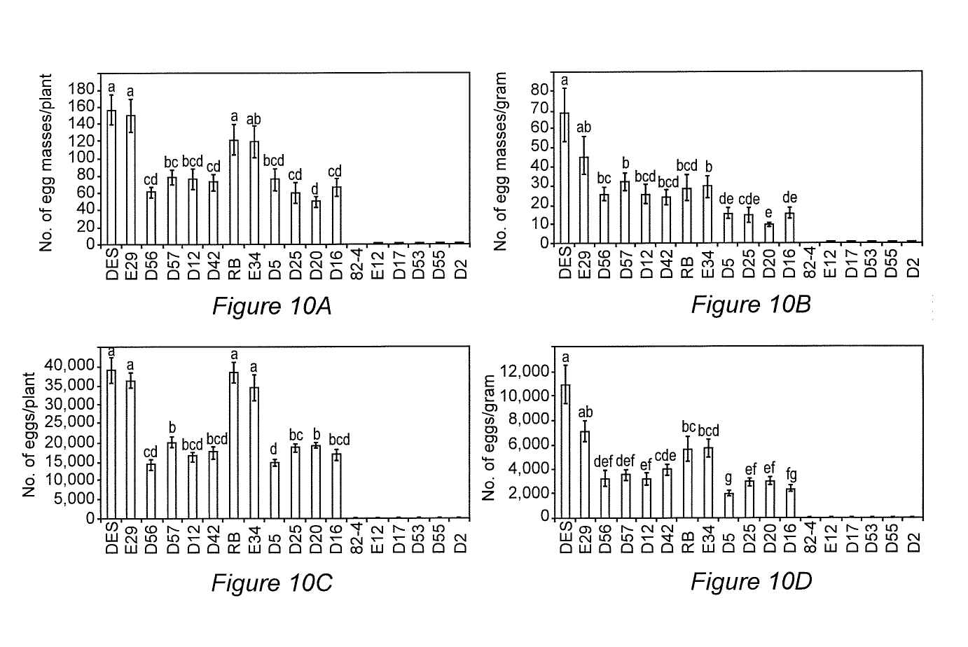

At 35 DAI with M. chitwoodi isolate WAMC1, the average number of egg masses per plant in cv. Désirée lines carrying 16D10i-2 was reduced significantly by 48-59% (P<0.05) compared to the empty vector control (FIG. 10). Similarly, the number of egg masses in lines of cv. Russet Burbank with 16D10i-2 were lowered significantly by 37-58% (P<0.05) relative to empty vector controls of the same genetic background. In general, there was neither a statistical difference among the 16D10i-2-carrying lines of either background, nor between the two cultivars for egg mass production. The only exception was cv. Russet Burbank line D20, which supported the smallest number of M. chitwoodi egg masses overall, thereby leading to a statistically significant difference relative to cv. Désirée D57 (P<0.05), the line with the greatest number of egg masses. Advanced breeding line PA99N82-4 and its transgenic 16D10i-2 derivatives did not support M. chitwoodi WAMC1 infection, and no more than two egg masses per plant were found in this background. At 55 DAI, the average number of eggs per plant was reduced significantly by 45-60% (P<0.05) in 16D10i-2 lines of cv. Désirée compared to the empty vector control. In cv. Russet Burbank, a significant reduction of 44-57% (P<0.05) was observed relative to the empty vector control line. Whereas the overall level of eggs per plant was similar within and between lines of cv. Désirée and cv. Russet Burbank, some lines were statistically different from others. For example, cv. Désirée D56 was significantly different from D57, the lines with the lowest and highest number of eggs, respectively. In addition, cv. Russet Burbank D5, which was the most resistant line in that background, showed a statistically significant difference compared to D20 and D25. Comparisons between both cultivars indicated significant differences for the least and most resistant lines, e.g., cv. Désirée D56 vs. cv. Russet Burbank D20 (all P<0.05). No eggs were found in control and transgenic lines of PA99N82-4. Comparable results were obtained when M. chitwoodi WAMC1 infection was expressed as average number of egg masses or number of eggs per gram root fresh weight, with statistically significant reductions of 29-48% and 44-55% compared to empty vector controls in cv. Désirée, and 47-68% and 47-65% in cv. Russet Burbank, respectively (P<0.05). Taken together, these experiments show that the 16D10i-2 RNAi transgene conferred a similar level of resistance against M. chitwoodi WAMC1 in both cv. Désirée and cv. Russet Burbank. Furthermore, 16D10i-2 did not interfere with the strong resistance against M. chitwoodi isolate WAMC1 that is mediated by the natural resistance gene R_{Mc1} and introgressed into line PA99N82-4.

RNAI Transgene 16D101-2 Increases Resistance Against M. chitwoodi Pathotype Roza

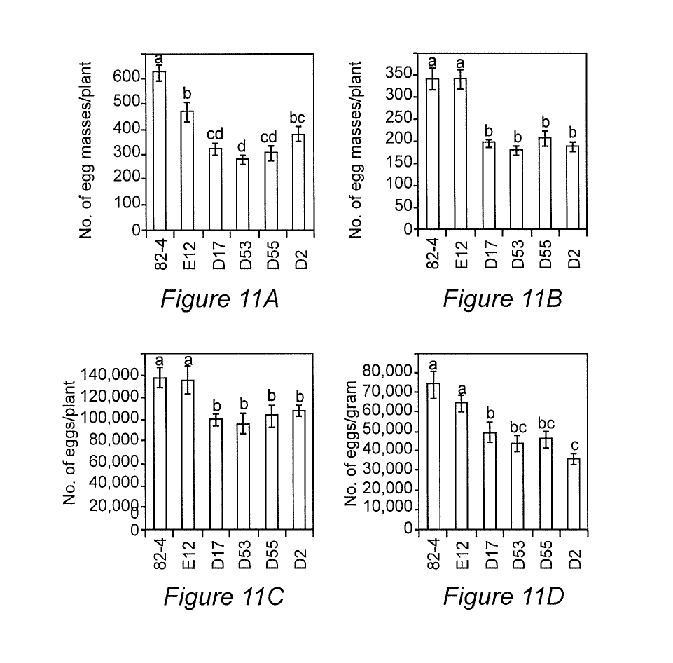

To analyze whether 16D10i-2 is able to provide an increased level of resistance against M. chitwoodi pathotype Roza, which is able to overcome the R_{Mc1} gene, PA99N82-4 and its transgenic RNAi derivatives were challenged with Roza following the same procedure. At 35 DAI the average number of egg masses per plant was reduced significantly by 32-40% (P<0.05) compared to the empty vector control (FIG. 11). One 16D10i-2 line, D2, showed a significantly lower number of egg masses relative to the wild type, but not relative to the empty vector control at P<0.05. Importantly, when expressed as egg masses per gram root fresh weight, all PA99N82-4 16D10i-2 lines, including D2, were statistically different from both the empty vector and wild type controls, and showed a reduction of 40-47% to either baseline. At 55 DAI the average number of eggs per plant was reduced significantly by 21-29% compared to the empty vector control, and resulted in a significant reduction of 23-44% when analyzed as eggs per gram root fresh weight (FIG. 11). These results demonstrate that 16D10i-2 significantly reduces the formation egg masses and the development of eggs of M. chitwoodi pathotype Roza.

RNAI Effect of 16D101-2 is Transmitted to M. chitwoodi Offspring and Reduces its Pathogenicity

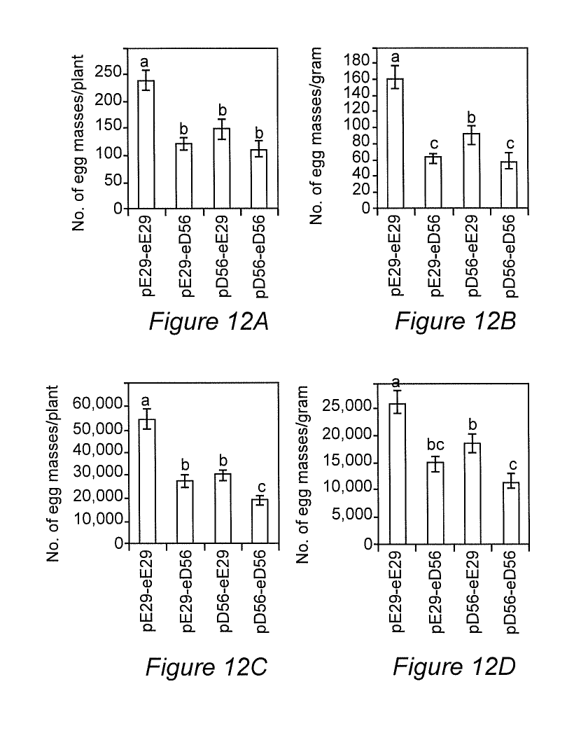

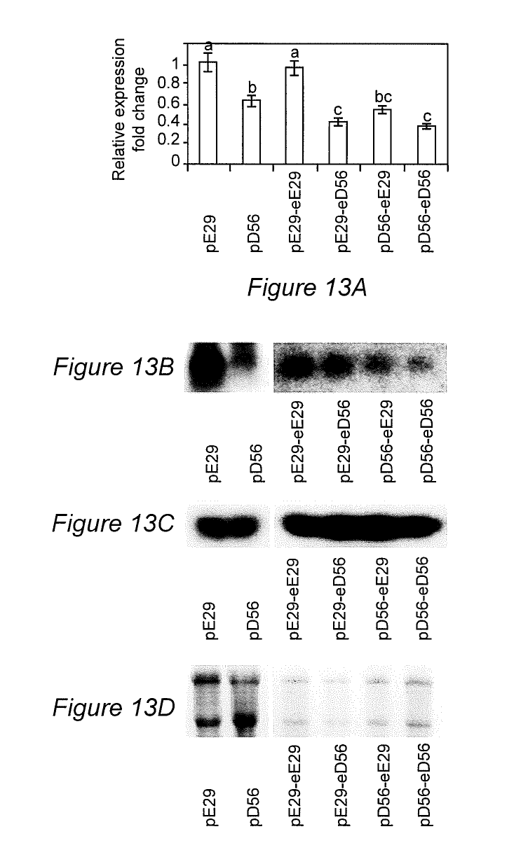

In order to examine whether 16D10i-2-mediated resistance has an effect on the pathogenicity of M. chitwoodi offspring, nematode eggs harvested from cv. Désirée carrying either the empty vector pART27 (designated as eE29) or the RNAi construct pART27(16D10i-2) (designated as eD56) were used to inoculate empty vector (pE29) or RNAi (pD56) plants, resulting in four possible plant-nematode combinations (see Materials and methods). Empty vector plants inoculated with M. chitwoodi eggs harvested from cv. Désirée carrying the empty vector pART27 served as baseline for all infection assay comparisons. Importantly, at 35 DAI the average number of egg masses per plant was reduced significantly by 49% (P<0.05) in empty vector plants inoculated with M. chitwoodi eggs harvested from cv. Désirée carrying 16D10i-2 (FIG. 12). Similarly, egg masses per plant were lowered significantly by on average 38% and 54% when potato plants expressing 16D10i-2 were inoculated with nematode eggs from empty vector and 16D10i-2 plants, respectively (P<0.05). At 55 DAI the number of eggs per plant was reduced significantly by 50% in empty vector plants inoculated with eggs from a 16D10i-2 line. Similarly, the number of eggs per plant was significantly lower (P<0.05) in cv. Désirée carrying 16D10i-2 inoculated with M. chitwoodi eggs from empty vector (−47%) or RNAi lines (−65%). Comparable results were obtained when the infection data were analyzed as egg masses or eggs per gram root fresh weight. Egg masses per gram root were reduced significantly by 62, 44 and 65% for plant-nematode combinations pE29-eD56, pD56-eE29 and pD56-eD56, respectively (P<0.05). Using gram root fresh weight metrics for the same host-inoculum combinations, the number of eggs was significantly lowered by 43, 30 and 56%, respectively (FIG. 12). This finding indicates that the RNAi effect of 16D10i-2 is transmitted to M. chitwoodi offspring, and significantly reduces its ability to complete its lifecycle.

To complement the infection data, the relative transcript level of the M. chitwoodi Mc16D10L effector gene, which is targeted by the 16D10i-2 RNAi construct, was analyzed by qRT-PCR and northern blots (FIG. 13). When 16D10i-2 plants were inoculated with nematode eggs from wild type plants, the transcript level of Mc16D10L in M. chitwoodi J2 offspring as detected by qRT-PCR was reduced significantly by 38% (P<0.05) relative to J2 from empty vector line pE29 using the same inoculum. Using the same baseline (Mc16D10L expression level in J2 from pE29 plants inoculated with eggs from wild type plants), the relative transcript level of Mc16D10L in J2 from pE29 plants and eE29 inoculum did not differ. Importantly, the transcript level of Mc16D10L was reduced significantly by on average 58, 46 and 62% (P<0.05) in J2 from plant-nematode combinations pE29-eD56, pD56-eE29 and pD56-eD56, respectively. This means that even in J2 from empty vector plants (pE29), which do not produce 16D10i-2 small RNAs, the transcript level of the Mc16D10L target gene was reduced by almost two thirds if the egg inoculum was harvested from 16D10i-2 plants (eD56). Northern blots confirmed the qRT-PCR results (FIG. 13).

### Discussion

Root-knot nematodes are a major problem for sustainable potato production in the Pacific Northwest of the USA. This study demonstrates that plant-mediated RNAi targeting of the putative effector gene Mc16D10L increases resistance against M. chitwoodi in stable transgenic lines of potato. RNAi is emerging as a promising molecular control strategy against plant-parasitic nematodes, but little is known about how to optimize the resulting resistance effect. For example, only a very limited number of studies have investigated whether changing the concentrations of small RNAs affects gene silencing in the nematode.

In this study the relationships between RNAi transgene copy numbers, small RNA concentrations and M. chitwoodi resistance were investigated in stable transgenic potato lines. Double or multiple insertions of 16D10i-2 do not necessarily lead to a higher level of 16D10i-2-specific small RNAs than single insertions. Furthermore, no significant difference in the level of resistance measured as the amount of egg masses or eggs was detected between plants that had single, double or multiple insertions of 16D10i-2. For example, cv. Désirée lines D12 and D42 with double and multiple insertions of 16D10i-2, respectively, did not differ in their small RNA level. It is conceivable that the signal in the northern blot was saturated, and disguised a possible small RNA concentration difference between D12 and D42. However, comparisons of other lines indicate that such was not necessarily the case. Russet Burbank lines D5 and D25 both had single transgene insertions, but showed a marked difference in their 16D10i-2-specific small RNA level. Factors other than the number of RNAi transgene insertions may have had an impact on the amount of small RNAs produced. One of the main aspects to be considered in this regard are position effects, which are a result of the random integration of transgenes into a genome. Position effects frequently are reported in potato and can override the regulatory control of the promoter associated with the transgene, such that the transcript level, as well as the temporal and spatial expression patterns of the transgene are altered. It has been shown that in A. thaliana methylation of the 35S promoter can cause epigenetic silencing of transgenic RNAi constructs, leading to strongly varying levels of small RNAs between lines. Thus, it is possible that in some cases one or more 16D10i-2 RNAi transgene copies were partially or fully deactivated in similar ways, thereby resulting in double or multiple insertion lines that produce small RNAs at a level equivalent to what would be expected in single insertion lines.

Previous studies aimed at creating stable transgenic RNAi plants to down-regulate nematode genes made use of a single genetic background of the recipient plant species. Solanum sect. Petota, which consists of wild and domesticated potatoes of tuber and non-tuber-bearing species, shows an exceptionally high level of genetic diversity. In this study an attempt was made to capture some of this diversity and examine whether it has an effect on in planta RNAi-mediated resistance against M. chitwoodi by introducing the 16D10i-2 transgene into cvs. Désirée and Russet Burbank, two different cultivars of domesticated potato (S. tuberosum ssp. tuberosum) and PA99N82-4, an advanced breeding line into which traits from the wild species S. bulbocastanum were introgressed. Regardless of the genetic background used, 16D10i-2-mediated resistance against M. chitwoodi reached comparable levels in all lines. This result suggests that there are no genotype-specific factors that would limit the use of RNAi in a broad range of germplasm.

In addition to genetic diversity of host germplasm used for resistance breeding, the variability of the pathogen needs to be considered. For M. chitwoodi, a system has been developed that distinguishes two races with one pathotype each, giving four different pathogenicity types that can be differentiated based on host assays with indicator plants (Brown et al., 2009; Humphreys-Pereira & Elling, 2013; Humphreys-Pereira & Elling, 2014). M. chitwoodi pathotype Roza is able to overcome the resistance gene R_{Mc1} that has been introgressed into breeding line PA99N82-4. When 16D10i-2 was introduced into PA99N82-4 and the resulting transgenic lines inoculated with M. chitwoodi isolate Roza, the number of egg masses and eggs per gram root were reduced by about 40%. This level of resistance is slightly less than what was found for M. chitwoodi isolate WAMC1 (race 1) in 16D10i-2 lines of cv. Désirée and cv. Russet Burbank. M. chitwoodi Roza is substantially more virulent than any other known isolate, with reproductive factors that can be ten times higher in Roza compared to WAMC1 (Brown et al., 2009). In this study, Roza produced five to seven times more egg masses and eggs on wild type control plants than WAMC1, even with the same amount of inoculum.

RNAi effects are known to be inherited in C. elegans, and maintenance of the RNAi phenotype for over 80 generations has been reported, but this phenomenon seems to be restricted to germline genes. In contrast, RNAi effects in Meloidogyne can be inherited even when autosomal genes that are not part of the germline are targeted. Here it is shown that M. chitwoodi that develop on 16D10i-2 potatoes transmit the plant-mediated RNAi phenotype to offspring, and that the RNAi effect is maintained for three generations even in the absence of selection pressure, and reduced the pathogenicity of the nematode offspring on empty vector control plants. Subsequent inoculation of 16D10i-2 plants with M. chitwoodi in which RNAi-mediated silencing of Mc16D10L already had been initiated, did not lead to a significantly increased level of resistance. This is an important finding, because it suggests that nematode infection decreases regardless of the genotype of the host once the RNAi process has been initiated through a transgenic plant. Thus, nematode resistance may be achieved in a field with mixed genotypes, as long as at least one genotype is transgenic and induces an RNAi phenotype in the nematode.

In summary, the experiments described here show that plant-mediated RNAi silencing of the putative effector gene Mc16D10L results in resistance against M. chitwoodi in diverse potato germplasm and that the RNAi effect is maintained over several generations, thereby providing resistance breeding programs with an effective new tool against this important pathogen.

### REFERENCES

Brown, C. R., Yang, C. P., Kwiatkowski, S. & Adiwilaga, K. D. (1991b). Insert copy number, chromosome-number, pollen stability, and crossability of Agrobacterium-transformed diploid potato. American Potato Journal 68, 317-330.
Brown, C. R., Mojtahedi, H., James, S., Novy, R. G. & Love, S. (2006). Development and evaluation of potato breeding lines with introgressed resistance to Columbia root-knot nematode (Meloidogyne chitwoodi). American Journal of Potato Research 83, 1-8.
Brown, C. R., Mojtahedi, H., Zhang, L. H. & Riga, E. (2009). Independent resistant reactions expressed in root and tuber of potato breeding lines with introgressed resistance to Meloidogyne chitwoodi. Phytopathology 99, 1085-1089.
Elling, A. A. (2013). Major emerging problems with minor Meloidogyne species. Phytopathology 103, 1092-1102.
Huang, G., Allen, R., Davis, E. L., Baum T. J. & Hussey, R. S. (2006a). Engineering broad root-knot resistance in transgenic plants by RNAi silencing of a conserved and essential root-knot nematode parasitism gene. Proceedings of the National Academy of Sciences USA, 103, 14302-14306.
Huang, G., Dong, R., Allen, R., Davis, E. L., Baum, T. J. & Hussey, R. S. (2006b) A root-knot nematode secretory peptide functions as a ligand for a plant transcription factor. Molecular Plant-Microbe Interactions 19, 463-470.
Humphreys-Pereira, D. A. & Elling, A. A. (2013). Intraspecific variability and genetic structure in Meloidogyne chitwoodi from the USA. Nematology 15, 315-327.
Humphreys-Pereira, D. A. & Elling, A. A. (2014). Morphological variability in second-stage juveniles and males of Meloidogyne chitwoodi. Nematology, in press (DOI:10.1163/15685411-00002753).
Hussey, R. S. & Barker, K. R. (1973). A comparison of methods of collecting inocula of Meloidogyne spp., including a new technique. Plant Disease Reporter 57, 1025-1028.
Livak, K. J. & Schmittgen, T. D. (2001). Analysis of relative gene expression data using real-time quantitative PCR and the 2^{-ΔΔCt} method. Methods 25, 402-408.
Porter, I., Banks, J., Mattner, S. & Fraser, P. (2009). Global phaseout of methyl bromide under the Montreal Protocol: Implications for bioprotection, biosecurity and the ozone layer. In: Gisi, U., Chet, I. & Gullino, M. L. (Eds). Recent developments in management of plant diseases. Plant pathology in the 21^{st} century. Berlin, Germany, Springer-Verlag, pp. 293-309.
Sheerman, S. & Bevan, M. W. (1988). A rapid transformation method for Solanum tuberosum using binary Agrobacterium tumefaciens vectors. Plant Cell Reports 7, 13-16.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A stable transgenic plant that is resistant to Meloidogyne chitwoodi, or a cell or plant part that is produced by or from said stable transgenic plant.

2. The stable transgenic plant of claim 1, wherein said stable transgenic plant expresses dsRNA comprising an RNA sequence that is complementary to at least a portion of an mRNA sequence that encodes a Meloidogyn effector protein.

3. The stable transgenic plant of claim 2, wherein said Meloidogyn effector protein is Mc16D10L.

4. The stable transgenic plant of claim 2, wherein said dsRNA is shRNA.

5. The stable transgenic plant of claim 1, wherein said stable transgenic plant is selected from the group consisting of potato, carrot, tomato, alfalfa, peas, beans, lucerne, carrots and black salsify.

6. The stable transgenic plant of claim 5, wherein said stable transgenic plant is a potato plant.

7. A method of causing a plant to be stably resistant to Meloidogyne chitwoodi comprising
genetically engineering said plant to contain and express dsRNA comprising an RNA sequence that is complementary to at least a portion of an mRNA sequence that encodes a Meloidogyn effector protein.

8. The method of claim 7, wherein said Meloidogyn effector protein is Mc16D10L.

9. The method of claim 7, wherein said dsRNA is shRNA.

10. The method of claim 7, wherein said plant is selected from the group consisting of potato, carrot, tomato, alfalfa, peas, beans, lucerne, carrots and black salsify.

11. The method of claim 10, wherein said plant is a potato plant.

12. A nucleic acid sequence encoding an shRNA comprising an RNA sequence that is complementary to at least a portion of an mRNA that encodes a Meloidogyn effector protein.

13. The nucleic acid sequence of claim 12, wherein said Meloidogyn effector protein is Mc16D10L.

14. The nucleic acid sequence of claim 12, wherein said nucleic acid sequence is present in a vector suitable for transforming a plant or plant cell.
